(19) United States
(12) Patent Application Publication
(10) Pub. No.: US 2015/0216918 A1
(43) Pub. Date: Aug. 6, 2015

(54) FERMENTED SOY NUTRITIONAL SUPPLEMENTS INCLUDING MUSHROOM COMPONENTS

(71) Applicant: Vijaya Nair, Bedford, NY (US)

(72) Inventor: Vijaya Nair, Bedford, NY (US)

(21) Appl. No.: 14/569,310

(22) Filed: Dec. 12, 2014

Related U.S. Application Data
(63) Continuation of application No. 13/032,504, filed on Feb. 22, 2011, now abandoned.
(60) Provisional application No. 61/305,994, filed on Feb. 19, 2010.

(51) Int. Cl. A61K 36/06; A61K 31/12; A61K 31/4525; A23L 2/52; A61K 36/068; A23L 1/28; A23L 1/303; A23L 1/302; A61K 36/48; A61K 36/074

(52) U.S. Cl. CPC A61K 36/06; A61K 36/48; A61K 31/12; A61K 31/4525; A61K 36/074; A61K 36/068; A23L 1/28; A23L 1/303; A23L 1/302; A23L 2/52; A23V 2002/00

(57) ABSTRACT

Dietary, health, and/or nutritional supplements provide various formulations of mushrooms grown in fermented soy, with or without rice flour, optionally in combination with sweetener(s), curcumin, and various other herbs and spices. Certain embodiments or compositions may be in liquid, beverage, solid, paste or powder forms. In certain embodiments the supplements contain mushroom species grown in fermented soy in the presence of certain bacterial species and optionally rice or rice flour. In some embodiments, other ingredients that may be present include one or more of curcumin, desmethoxycurcumin and bis desmethoxycurcumin or all three curcumins. Certain embodiments may be used to treat or provide symptomatic relief from a variety of maladies ranging from malnutrition to mood related disorders to metabolic support and other more severe conditions as described herein.

This patent application is a continuation under 35 U.S.C. 1.120 of U.S. Ser. No. 13/032,504, filed on Feb. 22, 2011, the contents of which are hereby incorporated by references for all purposes. This application claims the benefit of U.S. Provisional Application No. 61/305,994, filed Feb. 19, 2010, which is herein incorporated by reference.

### FIELD OF THE INVENTION

In connection with one or more non-limiting embodiments of the presently claimed invention, selected embodiments of formulations may include mushrooms grown in fermented soy in the presence of certain probiotic or prebiotic bacterial species, or similarly grown in fermented soy with the addition of rice or rice flour. In certain embodiments, piperine, curcumin and/or fermented soy may be added with other optional ingredients as described herein.

### BACKGROUND

The literature is replete with references to health benefits of certain herbs, spices, and natural food products. Approximately 75% of FDA approved drugs from 1981-2002 were either natural products, were based on natural products, or mimicked them in one form or another. Approximately 25% of the drug prescriptions in the United States are compounds derived from plants that were discovered through scientific investigation of folklore claims (Reynold, T., J. Natl. Cancer Inst., 1991 183: 594-596). A significant percentage of our current pharmaceuticals were obtained from isolation of natural products involved in longstanding traditional or folklore remedies.

Soy: Characteristics and Advantages

In the treatment of cancer and other diseases, patients frequently exhibit symptoms that interfere with their recovery even when there is clinical remission of the cancer. For example, malnutrition, anexoria, and cachexia are common problems encountered in cancer patients. Cachexia occurs in more than 80% of patients who have advanced cancer with symptoms of loss of appetite, chronic nausea, fatigue, and changes in body weight, lean body mass, and total body fat. Anorexia is associated with prolonged hospital stays and the length of the stay for rehabilitation. There are few effective treatments for the problem of cancer-associated cachexia. New treatment regimes to treat anorexia and cachexia in patients with advanced cancer are urgently needed. Soy isoflavones and its metabolites may be promising candidates for treatment cachexia associated with cancer. However, absorption of sufficient quantities of the relevant nutritional constituents will likely have to be achieved before true beneficial advantages from oral ingestion of soy containing compositions may be obtained.

Whole soybeans contain about 40% protein and are naturally high in isoflavones (including genistein, daidzein and their metabolites), phytosterols, saponins, protease inhibitors, phytic acid compounds, and the branched-chain fatty acid 13-methyl-tetradecanoic acid, that may provide positive nutritional benefits, including reduction of blood serum cholesterol levels among other health advantages. The active agent MTD-13 (13-methyl-tetradecanoic acid) may show some promise in causing cell death or apoptosis in tumor cell lines. Whether that translates into an anti-cancer agent remains to be fully validated.

Fermented soy products may provide a broader nutrient profile than non-fermented soy foods such as soy milk, soy nuts, tofu, and fresh or dry soybeans. Without being bound by theory, the soy fermentation process offers some hope in that fermented soy appears to increase the bioavailability of the constituents in soy by removing trypsin inhibitors (anti-nutritive or anti-nutrients in soy) found on the coating of soy. Such anti-nutrients interfere with the absorption of nutritive compounds. The benefits of fermented soy may include reducing risks of certain types of cancers, promoting heart and bone health, and alleviating menopausal symptoms—if the anti-nutritive soy components could be removed or rendered ineffective—so as to promote greater bioavailability and/or absorption of the nutritive constituents of soy, for example. Selected fermented soy products and methods are described in U.S. Pat. Nos. 4,877,739 and 7,070,965. However, it would be further beneficial to find compositions that promote beneficial absorption of nutrients in fermented soy and/or its co-constituent ingredients.

Although spices are generally added to food to improve taste, besides adding to taste they may have a variety of functions that may provide beneficial antimicrobial activity, antioxidant activity or functionality, and possibly a wide range of medicinal benefits. For example, in traditional folk medicine various herbs and spices have been used to treat digestive problems, including aiding digestion; relieving intestinal gas, constipation, diarrhea, dysentery; and even eliminating worms from the intestinal tract. While some medical claims for the efficacy of natural products have undoubtedly been exaggerated and others have proven false, many important medical claims are currently being investigated and some may be confirmed.

The variety of active compounds found in herbs and spices (i.e., phthallides, polyacetylenes, phenolic acids, flavonoids, coumarins, capsicinoids, triterpenoids, sterols, monoterpenes, alkaloids, etc) likely account for their activity. For example, antibacterial activity from dietary spices and medicinal herbs has been closely associated with their phenolic constituents [Shan, B., et al., International J. Food Microbiol, 117(1): 112-119 (2007)]. Herbs and spices linked to digestive and other health benefits may include, caraway and mustard seeds, juniper berries, spearmint, cinnamon, mace, pepper, ginger, coriander, fennel, cumin, nutmeg, sage, chocolate, garlic, dill, hyssop, oregano, rosemary, bee balm, lemon balm, sage, savory, thyme, parsley, bilberry, St. John's Wort, milk thistle, green tea, purslane, grape seed, cardamom, ginkgo, King. Solomon's. Seal, clove, stevioside, peppermint, licorice, sassafras, and turmeric. Cinnamon, for example, predominantly grown in. India, China and Sri Lanka, might provide a natural remedy against adult onset type II diabetes, lower “bad” LDL cholesterol, provide anti-blood clotting action, boost brain function and alertness, treat colds and flu, and act as a mild stimulant [Hunter, B., Consumers. Res. Mag., 85(10):8-8 (2002); Grey, L. W., Better. Nutrition, 69:18 (2007)]. Curcumin, the ingredient of turmeric that provides its yellow color, has shown promise in treating. Alzheimer's disease, and possibly cancer (i.e., being evaluated with ongoing research at M.D. Anderson. Cancer. Center in. Houston, Tex. and other cancer centers such as the Sloan-Kettering. Cancer. Center), and cystic fibrosis [Consumer. Reports, 71(1):49 [2006]. However, bio-absorption appears to be a hurdle to overcome before seeing the full range of certain benefits associated with curcumin ingestion. Ginger has a long history of medicinal use dating back over 2500 years. It is reported to help with motion sickness, loss of appetite, nausea, and may have anti-cancer properties attributed to certain vanilloids as well as other constituents like shogaols and zingerone [Shukla, Y., et al., Food. Chem. Toxicol., 45:(5):683-690 (2007)]. Raw ginger is reported to have hypoglycemic, hypocholesterolaemic and hypolipidaemic potential and may be of great value in managing diabetic complications [Al-Amin, Z. M., et al., Br. J. Nutr., 96(4):660-666 (2006)]. [6]-Gingerol, a naturally occurring plant phenol that is one of the main components of fresh ginger, has a variety of pharmacological effects, including some anti-oxidant, and/or some anti-inflammatory activities (i.e., transactivation of. Cox-2). Additionally, [6]-Gingerol may be an effective therapeutic agent providing protection against UVB-induced skin disorders [Kim, J. K., et al., Free. Radic. Res., 41(5):603-614 (2007)]. Peppermint is believed to help with loss of appetite, colds, coughs, bronchitis, sinusitis, fever, nausea and vomiting, indigestion and other digestive problems. Cardamom is reported to have antibacterial activity, including cavity-causing bacteria and bacteria that can cause dandruff and acne. Stevioside, a natural plant glycoside isolated from the plant. Stevia rebaudiana, has been used as a sweetener in Japan for more than 20 years and has been reported to have some anti-hypertensive effects [Chan, P., et al., Clin. Pharmacol., 50:215-220 (2000)]. Herbs are reported to be potentially important resources for drug candidates against the onset of Alzheimer's disease [Kim, D. S., et al., J. Altern. Complement. Med., 13(3):333-340 (2007)].

There is currently a demand for low-carbohydrate, low-sugar food products. Stevia (also called sweetleaf, sweet leaf or sugarleaf) is a genus of about 150 species of herbal shrubs in the sunflower family (Asteraceae), native to subtropical and tropical South America and Central America. As a natural sweetener, stevia has a slower onset but longer taste duration than sugar (sucrose), although some of its extracts may have a bitter or liquorice-like aftertaste at high concentrations. With its extracts having up to 300 times the sweetness of sugar, stevia has shown promise as a natural sweetener especially for diabetics and others on carbohydrate-controlled diets.

Among other chemical ingredients, many of the several thousand known herbs and spices may contain significant levels of antioxidants. The antioxidant properties of herbs and spices and their presumptive health and anti-aging effects, has led to the development and commercialization of a variety of nutritional herbal supplements, including some provided in pill form(s). Although the best way to get antioxidants and other beneficial food ingredients may well be through a diversified balanced diet, this is not often the case and there is strong contemporary need for convenient health foods providing nutritional benefits. The known antioxidant activity of dietary spices (clove, cinnamon, pepper, ginger, garlic, mint, onion, etc.) is one reason to suggest that, in addition to providing flavor to foods, they may provide potential health benefits.

Black pepper spice is obtained from peppercorns from fruits of Piper nigrum and is used widely throughout the world. Black pepper has been traditionally used in the Indian system of medicine ‘Ayurveda’ for the treatment of many ailments. Piperine (1-piperoylpiperidine), the alkaloid that is the active ingredient of black pepper, has various properties and uses, including flavoring, insecticide activity, anti-diarrheal activity, anti-inflaptmatory activity, potential ability to enhance the bioavailability of certain drugs, ability to stimulate the release of epinephrine, ability to elevate levels of circulating thyroid hormones, anti-oxidant functionality (i.e., ability to reduce high-fat diet induced oxidative stress to cells); and analgesic, antibacterial and antiseptic properties [Vijayakumar, R. S., et al., Redox Report, 9(2) 2004].

Dried long pepper is obtained from Piper longum. Long pepper is of South Asian origin and has a pungent and simultaneously sweet taste. It is more pungent than black pepper. In India, a major use for long pepper is in spicy vegetable pickles. Since terpene components are missing in the aroma of long pepper, it cannot be conveniently substituted for ordinary black pepper.

The Indian spice Turmeric, derived from the rhizome of the plant Curcuma longa, is a gold-colored spice traditionally used in the Indian Subcontinent for health care, the preservation of food, and as a yellow dye for textiles. Curcumin (diferuloylmethane) is the ingredient of turmeric that gives this spice its yellow color. Many therapeutic functions have been attributed to turmeric. Thus, curcumin may be useful for the treatment of a wide variety of diseases and conditions of the skin, pulmonary system, gastrointestinal system, aches, pains, wound, sprains, and liver disorders. Curcumin may have anti-oxidant, anti-inflammatory, anti-viral, anti-bacterial, anti-fungal, and/or anticancer activities, and may have potential use against various malignant diseases, diabetes, allergies, arthritis, Alzheimer's disease, and other chronic illnesses. Turmeric may be useful as an anti-inflammatory agent to treat gas, colic, toothaches, chest pains, and menstrual problems, to help with stomach and liver problems, to heal wounds and lighten scars. Turmeric may be used as a cosmetic. Turmeric contains various phytochemicals, including curcumin, demethoxycurcumin, bis-demethoxycurcumin, zingiberene, curcumenol, curcumol, eugenol, etrahydrocurcumin, triethylcurcumin, turmerin, turmerones, and tumeronols.

Given the foregoing, it will be appreciated that new nutritional formulations of natural plant products are desirable to provide dietary, health, and/or other benefits. Certain embodiments of the presently claimed invention are directed toward such goal with a focus towards enhancing bioavailability of constituents in mushrooms grown in fermented soy or grown in fermented soy with rice or rice flour.

Mushrooms: Characteristics and Advantages

Generally, a mushroom contains lower fat but higher protein and saccharide contents than any other plant. The saccharide also includes trehalose, mannitol, arabinose and the like together with polysaccharide that are hard to absorb into human intestinal tracts and of which main component is an indigestible dietary fiber. Therefore, mushroom is a food material having lower calories than those calculated by food analysis. Furthermore, ergosterol and calcium are commonly contained in an individual mushroom in an amount of 100-800 mg, and these components are converted into vitamin D₂ when dried. In addition to said ingredients, mushrooms contain vitamin B₁, vitamin B₂ and niacin, but not vitamins A and C. Also, as minerals, potassium (K) is included in a higher proportion than sodium (Na) therein, and phosphorus (P), calcium (Ca), and iron (Fe) follow in lesser amount. Flavorous ingredients of mushroom are mainly nucleic acid and a combination of glutamic acid, succinic acid, malic acid, uric alcohol, etc. Therefore, a mushroom is a food material not only having low calories and capable of exerting physiological functions, but also excellent in flavor, taste and gustatory quality.

However, mushroom material includes such large amounts of water and nitrogen compounds that it decays and microorganisms are easily propagated on and within the material due to its soft tissue. Accordingly, the shelf life after harvest of mushroom is short. Mushroom is distributed in the form of live or dried product. Because mushroom is used as a side dish or a flavoring, it plays a role in providing elementary nutrients.

Recently, mushroom has been known to an effective material for inhibition of cancer, for lowering of lipid in serum, for enhancing immunity to diseases, for inhibiting aging processes, and for preventing adult diseases. Furthermore, the use of mushrooms has been expanded into the medicinal field. Known medicinal mushrooms include Ganoderma lucidum, Lentinus edodes, Pleurotus osteratus, Elfvingia applanata, Agaricus, Auricularia auricula and Umbilicaria esculenta. In particular, it has been reported that polysaccharide protein complex included in Ganoderma lucidum extract exhibits the inhibition of the proliferation of cancer cells, treatment of essential hypertension, inhibition of the formation of peroxidized lipid, etc. Also, Lentinus edodes is well known to have anticancer properties, cholesterol-lowering effects, diuretic effects, and is used for treatment of hypertension, nephritis, asthma, gastric ulcer, etc. Its extract is reported to have effects for lowering lipid in serum and liver, and for inhibiting attendant liver damage. Also, the polysaccharide extract from Pleurotus osteratus was reported to have a cholesterol-lowering effect in serum, and an inhibiting effect against liver injuries caused by carbon tetrachloride. Furthermore, an extract obtained from fruit bodies and mycelia of Pleurotus osteratus was reported to have an antioxidative effect.

Accordingly, one or more embodiments described herein may provide new and improved formulations of plant products that supply nutritional benefits, health benefits or both.

One or more embodiments described herein may be provided in various liquid and solid forms as described herein. Certain embodiments may be provided as dietary nutritional supplements that may contain one or more of fermented soybean, curcumin and optionally additional herbs and spices together with mushroom(s) grown in fermented soy with certain bacteria that may produce lactic acid, together with mushroom species grown with rice or rice flour.

One or more embodiments described herein may provide dietary supplements that combine plant products in various forms that are convenient for human consumption and may contain mushroom(s) grown in fermented soy with or without rice flour in the presence of certain prebiotic or probiotic bacterial strains as described herein.

### SUMMARY OF THE INVENTION

One or more embodiments described herein provide a dietary supplement composition in powder (or other form) that may be a mixture of mushroom(s) grown in fermented soy, and curcumin, in amounts to provide nutritional or other benefits. Such embodiments may include fermented soy plus rice flour and other ingredients described herein. In an illustrative powdered supplement, the fermented soy may be provided in an amount from about 80 wt % to about 99.5 wt % and the curcumin may be provided in an amount from about 0.5 wt % to about 20 wt % based on the total weight of the composition. Preferably, curcumin is Curcumin C3 Complex®. The fermented soy powder is preferably obtained from dehydration of a liquid HAELAN 951® product (Stenotrophomonas fermentation product) or a comparable equivalent thereof such as SoyLac™. An alternative source of fermented soy (in powder or other suitable form) may be supplied by the product named SoyLac™ combining the genistein, daidzein and glycetein constituents thereof. The base dietary supplement powder of fermented soy and curcumin may further contain at least one sweetener ingredient selected from the group of: cyclamate, saccharine, aspartame, sulfame potassium, sucralose, sugar alcohols, stevioside, stevia, sucrose, glucose, maltose, luohan, xylitol and honey in an amount effective to provide a pleasant taste to the composition of interest. Preferably, the sweetener shall (in part) mask the pungent or bitter taste of curcumin (turmeric) and/or the unpleasant taste of fermented soy. Also, without being bound by theory, the growth of mushrooms in fermented soy (with or without rice flour) may also contribute to unpleasant taste. The sweetener, or mixture of sweeteners, may therefore be provided in an amount from about 0.5 wt % to about 2.0 wt % based on the total weight of the composition of interest. Suitable sweeteners are sucralose and stevia used individually or in combination. Other suitable sweeteners include xylitol and luohan.

In addition to a mushroom grown in a fermented soy base and combined with, the composition may also contain at least one other ingredient (plant, plant part, or any extract thereof) selected from the group of: the fermented soy itself, rice flour, turmeric (Curcuma longa), cumin (Cuminum cyminum), black pepper (Piper nigrum), long pepper (Piper longum), BIOPERINE®, cinnamon (Cinnamonum zeylanicum), coriander (Coriandrum sativum), red pepper (Capsicum frutescens), clove (Syzygium aromaticum), ginger (Zingiber officinale), tejpat (Cinnamon tamala), fennel (Foeniculum vulgare), Kaala Jeera (black cumin), cardamom (Amomum subulatum), cardamom long, kalonji (Nigella sativa), ajowan (Trachyspermum ammi), mint (Mentha piperta), sesame seeds (Sesamum indicum), asafetida (Ferula narthex), curry leaves (Murraya koenigii), pomergrate (Punica granatum), nutmeg (Myristicafragrans), saffron (Coccus sativus), dill (Anethum graveolens), oregano (Origanum vulgare), coriander (Coriandrum sativum), parsley (Petroselinum crispum), camphor (Cinnamomum camphor), licorice (Glycyrrhiza glabra), caraway (Carum carvi), mustard (Brassicajuncea), tamarind (Tamarindus indica), rosemary (Rosmarinus officinalis), onion (Alliumfistulosum), garlic (Allium sativum), almonds (Amygdalus comunis), cashews (Anacardium occidentale), walnut (Juglans nigra), mace (Myristicafragrans), mango (Magnifera indica), prunes (Prunus domestica), raisins (Vilis vinifera), pecans (Carya illinoensis), figs (Ficus carica), fenugreek (Trigonellafoneum-gracium), fenugreek seeds, flax (linum usitatissimum), til (Sesamum indicum), tulsi (Ocimum basilicum), lemon grass (Cymbogogon flexusosus), ginseng, green tea, lutein, ashwagandha, caraway seed, juniper berries, spearmint, mace, fennel, nutmeg, sage, hyssop, bee balm, lemon balm, sage, savory, thyme, bilberry, St. John's Wort, milk thistle, tea, green tea, purslane, grape seed, ginko, King Solomon's Seal, peppermint, sassafras, cocoa, cocoa powder, chocolate powder, lipids, lecithin, soy lecithin, carbohydrates (sugars, starches, etc.), fats (polyunsaturated, monounsaturated, or saturated), grains, malts, and barley malt and certain mushrooms described herein. Various other ingredients that may be included may be BIOPERINE®, Piper longum, ginger, cardamom long, cinnamon, soy lecithin, barley malt, natural chocolate flavor, cocoa, vanilla, and other flavors. The other ingredient(s) is/are preferably present in an amount from about 0.1 wt % to about 20 wt % based on the total weight of the composition.

The mushroom(s) grown in fermented soy together with the curcumin powder may also contain; (1) at least one sweetener ingredient selected from the group of cyclamate, saccharine, aspartame, sulfame potassium, sucralose, sugar alcohols, stevioside, stevia, sucrose, glucose, maltose, luohan, xylitol and honey in an amount from about 0.5 wt % to about 2.0 wt % based on the total weight of the composition; and (2) at least one other ingredient selected from: turmeric (Curcuma longa), cumin (Cuminum cyminum), black pepper (Piper nigrum), long pepper (Piper longum), BIOPERINE®, cinnamon (Cinnamonum zeylanicum), coriander (Coriandrum sativum), red pepper (Capsicumjrutescens), clove (Syzygium aromaticum), ginger (Zingiber officinale), tejpat (Cinnamon tamala), fennel (Foeniculum vulgare), Kaala Jeera (black cumin), cardamom (Amomum subulatum), cardamom long, kalonji (Nigella sativa), ajowan (Trachyspermum ammi), mint (Mentha piperta), sesame seeds (Sesamum indicum), asafetida (Ferula narthex), curry leaves (Murraya koenigii), pomergrate (Punica granatum), nutmeg (Myristicajragrans), saffron (Coccus sativus), dill (Anethum graveolens), oregano (Origanum vulgare), coriander (Coriandrum sativum), parsley (Petroselinum crispum), camphor (Cinnamomum camphor), licorice (Glycyrrhiza glabra), caraway (Carum carvi), mustard (Brassicajuncea), tamarind (Tamarindus indica), rosemary (Rosmarinus officinalis), onion (Alliumfistulosum), garlic (Allium sativum), almonds (Amygdalus comunis), cashews (Anacardium occidentale), walnut (Juglans nigra), mace (Myristica jragrans), mango (Magnifera indica), prunes (Prunus domestica), raisins (Vilis vinifera), pecans (Carya illinoensis), figs (Ficus carica), fenugreek (Trigonellafoneum-gracium), fenugreek seeds, flax (linum usitatissimum), til (Sesamum indicum), tulsi (Ocimum basilicum), lemon grass (Cymbogogon flexusosus), ginseng, green tea, lutein, ashwagandha, caraway seed, juniper berries, spearmint, mace, fennel, nutmeg, sage, hyssop, bee balm, lemon balm, sage, savory, thyme, bilberry, St. John's Wort, milk thistle, tea, green tea, purslane, grape seed, ginko, King Solomon's Seal, peppermint, sassafras, cocoa, chocolate, lipids, lecithin, soy lecithin, carbohydrates (sugars, starches, etc.), grains, grain malts, and barley malt, in an amount from about 0.1 wt % to about 20 wt %, based on the total weight of the composition. Preferred other ingredients may be BIOPERINE®, Piper longum, ginger, cardamom long, cinnamon, soy lecithin, barley malt, natural chocolate flavor and cocoa. Also, see certain constituents/ingredient(s) recited in the appended claims.

In the powder dietary supplement containing a mushroom (grown in fermented soy with or without rice flour), optionally containing fermented soy, curcumin, a sweetener and other ingredients, the fermented soy component may be fermented soy powder obtained as HAELAN 951® or as SoyLec™; the curcumin ingredient may be Curcumin C3 Complex®; the at least one sweetener ingredient may be luohan, xylitol or a mixture thereof, or it may be xylitol alone or another monosaccharide or a mixture of stevia and sucralose, for example; and another at least one additional ingredient may be a mixture of BIOPERINE®, Piper longum, ginger, cardamom long, cinnamon, barley malt, soy lecithin, cocoa, and/or chocolate as a flavorant.

One or more illustrative embodiments of the presently claimed invention may be in the form of a dietary supplement containing (i) fermented soy; (ii) curcumin; (iii) at least one sweetener ingredient selected from the group of cyclamate, saccharine, aspartame, sulfame potassium, sucralose, sugar alcohols, stevioside, stevia, sucrose, glucose, xylitol, luohan, maltose and honey, and any combinations or subcombinations thereof; (iv) at least one additional plant ingredient, plant part, or any extract thereof, selected from the group of: mushrooms grown with a prebiotic or probiotic bacteria in fermented soy, or grown in fermented soy with rice flour, turmeric (Curcuma tonga), cumin (Cuminum cyminum), black pepper (Piper nigrum), long pepper (Piper longum), BIOPERINE®, cinnamon (Cinnamonum zeylanicum), coriander (Coriandrum sativum), red pepper (Capsicumfrutescens), clove (Syzygium aromaticum), ginger (Zingiber officinale), tejpat (Cinnamon tamala), fennel (Foeniculum vulgare), Kaala Jeera (black cumin), cardamom (Amomum subulatum), cardamom long, kalonji (Nigella sativa), ajowan (Trachyspermum ammi), mint (Mentha piperta), sesame seeds (Sesamum indicum), asafetida (Ferula narthex), curry leaves (Murraya koenigii), pomergrate (Punica granatum), nutmeg (Myristicafragrans), saffron (Coccus sativus), dill (Anethum graveolens), oregano (Origanum vulgare), coriander (Coriandrum sativum), parsley (Petroselinum crispum), camphor (Cinnamomum camphor), licorice (Glycyrrhiza glabra), caraway (Carum carv!), mustard (Brassicajuncea), tamarind (Tamarindus indica), rosemary (Rosmarinus officinalis), onion (Alliumfistulosum), garlic (Allium sativum), almonds (Amygdalus comunis), cashews (Anacardium occidentale), walnut (Juglans nigra), mace (Myristicafragrans), mango (Magnifera indica), prunes (Prunus domestica), raisins (Vilis vinifera), pecans (Carya illinoensis), figs (Ficus carica), fenugreek (Trigonellafoneum-gracium), fenugreek seeds, flax (linum usitatissimum), til (Sesamum indicum), tulsi (Ocimum basilicum), lemon grass (Cymbogogon flexusosus), ginseng, green tea, lutein, ashwagandha, caraway seed, juniper berries, spearmint, mace, fennel, nutmeg, sage, hyssop, bee balm, lemon balm, sage, savory, thyme, bilberry, St. John's Wort, milk thistle, tea, green tea, purslane, grape seed, ginko, King Solomon's Seal, peppermint, sassafras cocoa, chocolate, lipids, lecithin, soy lecithin, carbohydrates (sugars, starches, etc.), grains, and grain malts (e.g., barley malt); and (v) a pharmaceutically, nutraceutically, or cosmetically acceptable carrier. Certain other ingredients may include BIOPERINE®, Piper longum, ginger, cardamom long, cinnamon, soy lecithin, barley malt, natural chocolate flavor and cocoa. A pharmaceutically, nutraceutically, or cosmetically acceptable carrier or solvent may be conveniently selected from the group of water, aqueous systems, tea, green tea, coffee, fruit juices, non citrus juices, glycerin, alcohols, fatty alcohols, fatty ethers, fatty esters, polyols, glycols, vegetable oils, mineral oils, fats (polyunsaturated, monounsaturated, or saturated), cultured dairy products, dairy products, carbonated or non-carbonated beverages. See other permutations recited in the appended claims and additional embodiments described herein.

In one embodiment in accordance with the presently claimed invention, such embodiment may contain one or more mushroom species grown in fermented soy. Such embodiments may include curcumin, desmethoxy curcumin, and/or des-methoxycurcumin. This embodiment may further include at least one sweetener, and at least one additional ingredient. Also, this embodiment may optionally contain fermented soy in an amount from about 0.5 wt % to about 90 wt % based on the total weight of the final composition. Likewise, the curcumin may be present in an amount from about 0.5 wt % to about 20 wt % based on the total weight of the final composition. The sweetener may be present in an amount from about 0.1 wt % to about 2.0 wt % based on the total weight of the final composition. Additionally, the at least one additional ingredient (plant, plant part, or extract thereof) such as the mushroom species may be present in an amount from about 0.1 wt % to about 20 wt % based on the total weight of the final composition of interest. Such embodiments may further include a carrier or a solvent (e.g., the solvent being a beverage containing the embodiment of interest) in an amount from about 0.5 wt % to about 90 wt % based on the total weight of the composition. This supplement may be in a product form selected from the group consisting of a liquid, an emulsion, a suspension, a dispersion, a dispersion, a solid, a powder, a solution, a beverage, a smoothie, a shake, a juice, a tablet, a bakery item, and a capsule or a caplet, among others. The fermented soy ingredient (if present in a given embodiment of interest) may be HAELAN 951® or powder form of fermented soy obtained from HAELAN™ in an amount from about 0.5 wt % to about 90 wt % based on the total weight of the final composition of interest. The curcumin may be the Curcurnin C3 Complex® in an amount from about 0.5 wt % to about 20 wt % based on the total weight of the final composition of interest. The at least one sweetener ingredient may be a mixture of stevia and sucralose or a mixture of luohan and xylitol provided in an amount from about 0.1 wt % to about 2.0 wt % based on the total weight of the final composition of interest. If included, at least one additional ingredient may be an admixture of a mushroom species grown in the fermented soy, or grown in fermented soy with rice flour and with probiotic or prebiotic bacteria, optionally together with ingredients selected from the group consisting of BIOPERINE®, Piper longum, ginger, cardamom long, cocoa, chocolate, barley malt, cinnamon, and combinations thereof. The mushroom species may be present in an amount from about 0.1 wt % to about 20 wt based on a total weight of the final composition of interest. The carrier may be water in an amount from about 0.5 wt % to about 90 wt %, based on the total weight of the composition or embodiment of interest.

A powder form of fermented soy may be included in selected embodiments and may be obtained from liquid fermented soy by a method of heating liquid fermented soy under vacuum to remove water and to produce a concentrated fermented soy composition; vaporizing the concentrated fermented soy composition under pressure; and exposing said vaporized soy composition to hot air thus further removing remaining water from the soy composition and ultimately producing the powder form of the fermented soy if sought to be included in an embodiment as an optional ingredient or even if just intended to be included as the growth medium for the mushroom species described herein. A liquid fermented soy may also be used such as HAELAN 951®, or equivalent thereof. For example, instead of HAELAN 951®, it may be desirable to use a different fermented soy product such as SoyLac™ for a variety of reasons where a product free or essentially free of HAELAN 951® is desired or sought.

### BRIEF DESCRIPTION OF THE DRAWINGS

FIG. 1 describes several embodiments of therapeutic applications of combination of curcumin and fermented soy used in accordance with an embodiment of the present invention.

FIG. 2 describes some of the major ingredients of whole soybeans, or products produced from soybeans, as contemplated in several embodiments of the present invention.

### DETAILED DESCRIPTION

As used herein, the term “formulation” or the term “formulations” may include mushroom species grown in fermented soy with probiotic or prebiotic bacteria, optionally with rice flour included in the growth medium. Such formulations may include curcuminoids. Such formulations may include carriers, excipients, solvents, flavors, or any other ingredients as further described herein. For common excipients and carriers, see Remington's Pharmaceutical Sciences, infra, listing various pharmaceutical excipients, diluents, additives, carriers, lubricants, and the like.

As used herein, the term “animal” includes any living organism characterized by voluntary movement.

As used herein, the term “subject” may include a “human” and/or an “animal”, unless specified otherwise.

As used herein, the term “%” or “wt. %” is a percent by weight value based on a total weight (taken as 100 wt. %) of the formulation with all of its constituent ingredients accounted for, such as listed ingredients (active and/or inactive), excipients (if any), carriers (if any), or active pharmaceutical ingredients (if any), unless indicated otherwise.

As used herein, the term “organic” may include ingredients that may be farmed or grown without the use of certain pesticides, antibiotics and/or genetically altered or genetically modified plants, seeds, or organisms.

As used herein, the term “non-organic” may include ingredients that may be farmed or grown, optionally with the use of pesticides, antibiotics and/or genetically altered or genetically modified plants, seeds, or organisms.

As used herein, the term “treatment” may include care provided to improve a condition or alleviate some discomfort or undesirable consequence of the condition such as a chronic disease or condition, or even an acute disease or condition or illness, etc.

As used herein, the term “symptomatic relief” may include control or alleviation of symptoms and/or side effects obtained with the aid or ingestion of one or more embodiments of the presently claimed invention.

According to one or more illustrative embodiments, formulation(s) in connection with the presently claimed or described invention(s) may include one or more of the numerous constituents described herein or recited in the claims.

Furthermore, various permutations of the order of addition may be employed in connection with making one or more embodiment(s) or one or more formulation(s) in connection with the presently claimed or described invention(s).

Any one or more of the formulation(s) or embodiment(s) thereof described herein may have varying ingredient contents. Thus, for example, the amount of each ingredient may fluctuate within a certain range (but not limited to): about ±1%, up to about ±10%, as recited herein or in the appended claims. The various embodiments may also include well-known excipients, carriers, fillers, pigments, colorants, preservatives, diluents, solvents, and emulsifiers suitable for use in formulations, e.g. topical. Such embodiments may optionally include active ingredients in various forms, including, but not limited to, liquids, powders, syrups, shakes, tablets, capsules, gelatin capsules, concentrates, pills, cut strips, gum strips, dissolvable strips, emulsions, and the like.

Embodiments in accordance with the presently claimed invention may provide dietary supplementation, nutritional supplementation, promote synergistic absorption of certain constituents, promote enhanced activity of certain constituents, promote or provide superior symptomatic relief, treatment or some other health benefit from the combination of the constituent ingredients as opposed to that of the constituent ingredients being administered separately. Certain compositions or formulations of the presently claimed invention may comprise mushrooms grown in fermented soy, or alternatively, mushrooms grown in fermented soy with rice or rice flour. These compositions or formulations may further include curcumin, desmethoxycurcumin, bisdesmethoxycurcumin and an independently optional selections of various herbs, spices, and natural or processed ingredients as further described herein.

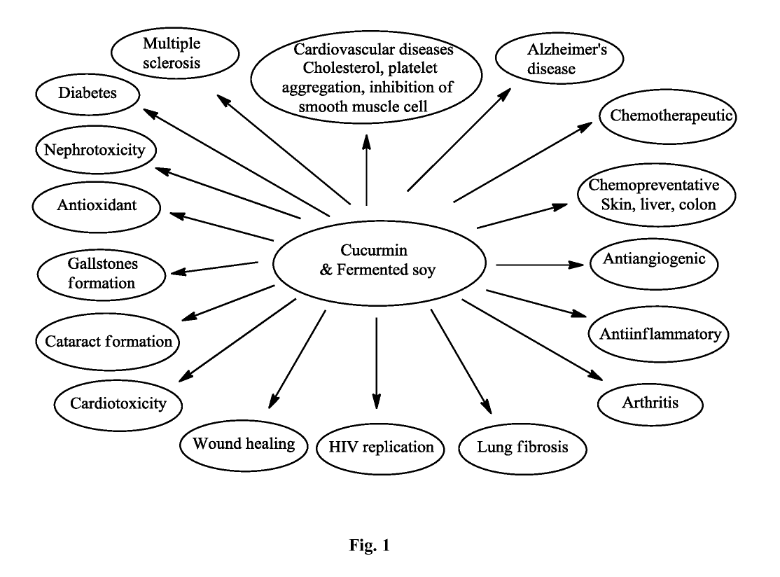

Referring now to FIG. 1, illustrated are some of the potential therapeutic applications of curcumin and fermented soy including, but not limited to, diabetes, multiple sclerosis, cardiovascular diseases (cholesterol, platelet aggregation, inhibition of smooth muscle cell), Alzheimer's Disease, chemotherapeutics, chemoprevention (skin, liver, colon), antiangiogenics, anti-inflammatories, arthritis, lung fibrosis, HIV replication, wound healing, cardiotoxicity, cataract formation, gall-stone formation, antioxidants, and nephrotoxicity.

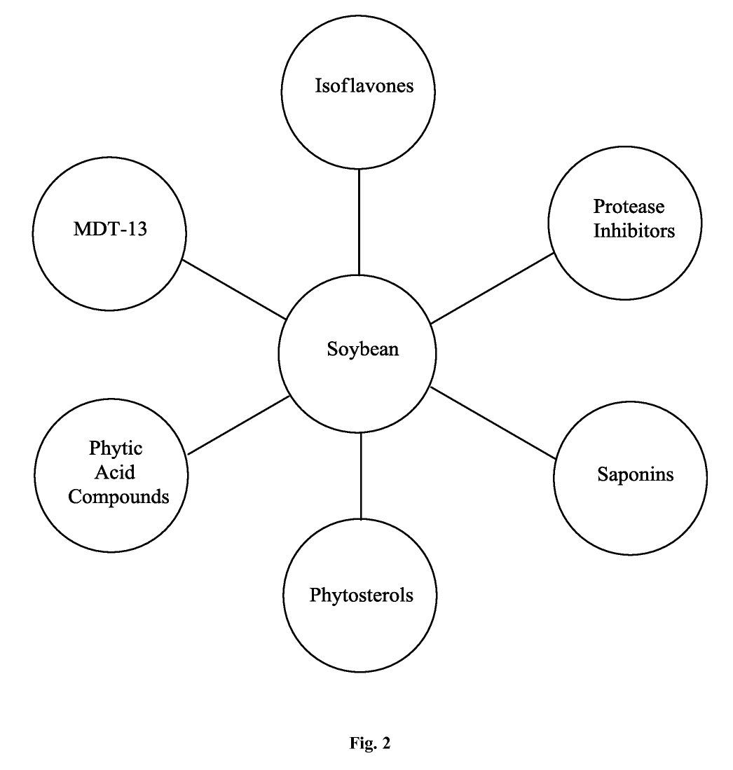

Referring now to FIG. 2, illustrated are the ingredients of whole soybean including isoflavones (including genistein, daidzein and their metabolites), protease inhibitors, saponins, phytosterols, phytic acid compounds, and the saturated branched-chain fatty acid MDT-13 [13-methyl-tetradecanoic acid]. The active agent MDT-13 has been shown to induce apoptosis in tumor cells. Research has shown that the nuclear factor kappaB (“NF-KB”) family of transcription factors are involved in multiple cellular processes, including cytokine gene expression, cellular adhesion, cell cycle activation, apoptosis and oncogenesis. Nuclear factor (NF-KB) has been shown to regulate proliferation, invasion, and metastasis of tumor cells. In animal models, suppression of NF-KB has shown decrease of cancer associated cachexia. Soy isoflavone, especially genistein, has been shown in animal studies to promote apoptosis by decreasing NF-KB activity.

Fermentation may be the oldest known form of food biotechnology, and records of barley conversion to beer date back more than 5000 years. The traditional fermentation process serves several functions, including the enrichment of food substrates biologically with protein, essential amino acids, essential fatty acids, vitamins, polyamines, carbohydrates, and numerous anti-oxidants, and phytosterols. An alternative fermentation process, for example, with lactobacilli, increases the quantity, availability, digestibility, and assimilation of nutrients in the body.

Without intending to be bound by theory, is it hypothesized that certain naturally fermented soy food beverages (e.g., HAELAN 951®, SoyLac™, or a combination thereof) may improve the cachexia, anorexia, and fatigue symptoms as well as enhance the nutritional status of patients or individuals with various serious diseases or conditions (e.g., advanced cancer, AIDS, geriatric patients, malnutrition, HIV infection, bacterial infection, viral infection, and the like). The fermentation process may improve nutrient availability from soy. Further, growth of mushroom species in fermented soy may improve the nutrient availability of certain mushroom constituents. The fermented soy may contain 12- and/or 13-methyltetradecanoic acids (“12-MTD”/“13-MTD”). Researchers at MD Anderson Cancer Center (Houston, Tex.) and other research centers have found that that targeted arterial delivery of branched chain fatty acids such as 12-MTD may be effective for treatment of solid tumors. The exact mechanism(s) responsible for inhibition of tumor growth in animal models by 12-MTD is unclear. Additional in vivo studies are warranted to elucidate 12-MTD's mechanism of action and further investigate the branched chain fatty acid's antitumor effect potential and its benefits against its risks.

Three fermented soy beverages are available or have been sold in the U.S. market, namely, EcoNugenics®, Soy Unique™, and HAELAN™. Of these three products, a market report analysis done by Iowa State University in May 2000 showed that the soy isoflavones, daidzin, genistin, and as well as its fermented isofloavone metabolites were significantly higher in the HAELAN 951® samples as compared to the other two products. HAELAN 951® may be a preferred fermented soy product for preparation of the concentrated soy protein powder used in formulations of one or more embodiments of the presently claimed invention in some instances. The dietary nutraceutical HAELAN 951®, a fermented soy supplement, is made by HAELAN 951® Products Inc. based in Seattle, Wash., USA. HAELAN 951® is a cultured soybean product manufactured by bacterial fermentation as described in U.S. Pat. No. 7,070,965, herein incorporated by reference. Another fermented soy product by the designation SoyLac™ may be beneficial in certain other embodiments in accordance with the presently claimed invention.

The manufacture of HAELAN 951® hydrolyzes many of the soybean proteins into amino acids and compounds that are rich in nitrogen, polysaccharides, and fermentation metabolites of the naturally occurring isoflavones, protease inhibitors, saponins, phytosterols, and inositol hexaphosphate compounds in soybeans. The manufacturing process (by fermentation) of HAELAN 951® uses the bacterium Stenotrophomonas maltophilia Q-can. This soy beverage is a concentrated nutritional supplement that is rich in soybean proteins, selenium, zinc, beta-carotene (Vitamin A), riboflavin (Vitamin B1), thiamine (Vitamin B2), cyanocobalamin (Vitamin B 12), ascorbate (Vitamin C), cholecalciferol (Vitamin D3), alpha tocopherol (Vitamin E), and phylloquinone (Vitamin K1). It also contains additional micronutrients such as daidzein, genistein, protease inhibitors, saponins, phytosterols, inositol hexaphosphate, essential fatty acids such as linolenic and linoleic acids, polysaccharides, peptides, and twenty-two amino acids including ornithine. It takes over 25 pounds of soybeans to make one concentrated eight-ounce-bottle of HAELAN 951® beverage. Of these naturally occurring nutritional items in the HAELAN 951® formula, more than twenty of them are known individually for their ability to enhance the nutritional profiles of individuals with cancers. The fermentation process breaks down the complex proteins of soybeans, making them more easily digested than the proteins of whole soybeans. Soy fermentation also serves to activate soy isoflavones. Fermented soy products contain more unconjugated isoflavones than non-fermented soy products due to microbial action during fermentation.

Experimental results with HAELAN 951® have shown that this composition stimulates and accelerates the hematopoiesis of infant mice; lowers oxidative stress reactions (i.e., reduction of hepatic and cerebral lipoperioxide concentrations); and provides immunological benefits (i.e., enhances cellular immunity). Some evidence suggests that it may protect against acute hepatic damage; and may provide anti-mutating benefits in cancer by resisting base substitution or by some other mechanism.

Stenotrophomonas maltophilia, used to ferment soy producing HAELAN 951®, contains a high percentage of branched-chain fatty acids (cf., U.S. Pat. No. 7,070,965). Other bacterial genera containing high percentage branched-chain fatty acids are: Xanthomonas, Flavobacterium, Capnocytophga, Altermonas, Cytophage, Bacillus, Chryseobacterium, Empdobacter, Aurebacterium Sphinggobaacterium, Staphylococcus, Azobacter and Pseudomonas. These bacteria, and others that may contain a high percentage of branched chain fatty acids (“BCAAs”), are microorganisms that may be considered for fermentation of soybeans for use in the preparation of compositions or formulations in accordance with one or more embodiments relating to the presently claimed invention. For example, Azotobacter vinelandii is a diazotroph described in U.S. Pat. No. 4,877,739 that can fix nitrogen while grown aerobically. It is a genetically traceable system that is used to study nitrogen fixation. These bacteria are easily cultured and grown. It is a free-living nitrogen fixer which is known to produce many phyto-hormones and vitamins in certain soils (O. Pseudomonadles).

Without intending to be bound by theory, it is slowly being increasingly recognized that soy and soy protein isolates may have potentially anti-nutritive value due to high phyate and oxalic acid content levels. In contrast, cultured soy products such as miso, natto, and tempeh have enhanced nutritive bioavailability without the possible ill effects of the uncultured soy supplements. Again, without being bound by theory, uncultured soy products with high phyate levels appear to block absorption of vital nutrients such as calcium. The use of soy as a culturing medium is advantageous because iron is naturally available in soy. Iron in an elemental form is potentially toxic and pro-oxidative, but a lack of iron in the body potentiates development of anemia and can thus be lethal. Cultured soy appears to enhance the bioavailability of iron and copper, and hence providing delivery of these nutrients in their most advantageous forms.

The World Health Organization reported in 2000 that the Japanese population with its extensive consumption of cultured soy products, such as miso and natto, together with accompanying foods like ginger, ocean herbs, and green tea, have the longest and “healthiest life expectancy” of any people on Earth. Americans, on the other hand, are not even in the top 20 list of the longest healthy life expectancy of any developed nation. The Western culture and diet places undue emphasis on the consumption of “smart” products that are processed or genetically modified. Unfortunately, the abandonment by consumers in developing countries of their more traditional fermented foods in exchange for the “sophisticated” Western dietary products could have serious consequences. For example, the replacement of indigenous fermented cereal drinks with cola beverages that have “empty” or excess calories could have a serious impact on the daily nutrition of many of these consumers in developing countries when the extensive medical benefits of consuming the traditional sources of probiotic whole-food nutrition is lost. However, such lost nutrition may be replenished even in a beverage diet with proper supplementation added in the beverage or other product of interest.

Soy has been a staple of the Southeast Asian diet for nearly five thousand years. Both its medicinal and nutritional values are deeply rooted in Traditional Chinese Medicine and herbalism. On the other hand, consumption of soy in the United States and Western Europe has been limited to the 20th century. Asians on the average eat twenty to fifty times more soy than Americans.

Typically, soy foods are divided into two categories: non-fermented and fermented soy products. Traditional non-fermented soy foods include fresh green soybeans, whole dry soybeans, soy nuts, soy sprouts, whole-fat soy flour, soymilk, and soymilk products such as tofu, okra, and yuba. Traditional fermented soy foods include tempeh, miso, soy sauces, natto, and fermented tofu and certain soymilk products. In Asia, the traditional fermented soy foods are considered to have more health-promoting benefits when consumed in moderate amounts than the super-processed soy products that are consumed in the West. Without intending to be bound by theory, soy fermentation may increase availability of isoflavones in soy. Miso, a fermented or probiotic form of soybean, is particularly rich in the isoflavone aglycones genistein and daidzein, which are believed to be cancer chemopreventives. The fermentation process is thought to convert the isoflavone glycoside precursors genistin and daidzin to their active anti-cancer isoflavone forms, genistein, and daidzein, respectively. It is unfortunate that in the United States, health-conscious consumers, especially women concerned about developing breast cancer, are urged by media and consumer reports to consume soy or soy protein isolates, which are not the forms in which soy is traditionally consumed in Japan. The overwhelming majority of soy consumed in Asian countries such as Japan, China, Korea, and Indonesia is in its cultured or probiotic form, enhanced with genistein and daidzein. Japanese researchers have found that the cultured broth of Saccharomyces cerevisiae, or Brewer's Yeast, produces both in vivo and in in vitro experimental results demonstrating potentially antimutagenic and anti-clastogenic activity with mutagen formation reduced by 47% just by the administration of the cultured broth. Without intending to be bound by theory, it is hypothesized that cultured soy medium may have the potential to produce its own unique anti-carcinogenic activity by strongly inhibiting formation of cellular mutations. The Chinese appear to believe in the health benefits of fermented soy foods (e.g., Douchil salted black bean, soy sauce etc.).

A recent Korean study concluded that tofu was one of the foods that decreased the rates of certain cancers, such as stomach cancer, while heavy salt consumption and cooking methods increased the rates of cancer. The limited epidemiological evidence obtained so far from cultures with diets that are high in fermented soy products may suggest that such diets may reduce risk of breast, colon, lung, and stomach cancers, as well as offer some protection from cardiovascular diseases, osteoporosis, and menopausal symptoms. However, the clinical data to date is often flawed, with attending poor design and other methodological issues. Consequently, the role of isoflavones in chemoprevention, especially in the hormonally-dependent cancers, such as breast and prostate cancers, is still lacking unequivocal supporting evidence.

The controversy over the role of soy in preventing and treating chronic illnesses such as cancer was recently addressed at the 4^{th} International Symposium held in San Diego, Calif. in November 2001. To date, there has been a paucity of literature regarding soy, particularly the use of fermented soy products in clinical trials. Part of the reason may be the costly technology required in processing and manufacturing these fermented soy products and dietary/nutritional supplements for large-scale use. Studies have been performed in order to determine if the phytoestrogens content of these products are affected by processing, or whether there are chemical modifications of the isoflavonoids due to the conditions present during the fermentation processing. Traditional sources of fermented soy foods that are not prepared under stringent standards lack appropriate control to make any sound evaluation. Tempeh production, for example, requires a fermentation process utilizing whole soybeans, but there have been few clinical reports on the effects of its chemically modified isoflavonoids.

Some studies have suggested that the isoflavone genistein may have some role as a chemopreventive agent against cancer in humans. Levels of genistein and its beta-glucoside conjugate, genistin, ingested in soybeans and related bean products by the Japanese were quantified by HPLC, to estimate daily intake of these compounds. A Japanese study found that the level of genistein in the fermented soybean products was higher than in soybeans and soybean products such as soymilk and tofu. Such studies have postulated that the beta-glycosyl bond of genistin is cleaved to produce genistein by microbes during fermentation to yield miso and natto. Soy sauce was also found to contain both isoflavones, but at the levels lower than in miso and natto. On the basis of these data for average annual consumption of soybeans and related products, daily intake of genistein and genistin by the Japanese was calculated to be 1.5-4.1 and 6.3-8.3 mg/person, respectively. These levels are much higher than those of Americans or Western Europeans, whose mortality rates for breast, colon, and prostate cancers are greater than the Japanese. Therefore, a correlation may exist between consumption of isoflavonoids from soy products at certain levels and cancer prevention.

Again without being bound by theory, one underlying theoretical paradigm may be that primary active ingredients in complex fermented soy “foods” act synergistically with secondary compounds. Likewise, without being bound by theory, a second paradigm may be that secondary compounds mitigate the undesirable side effects caused by the predominant active ingredients. Another possibility is that the multiple ingredients act through multiple discrete pathways to therapeutically affect the host or individual. Under another hypothesis, lower concentrations of each of the botanicals or soy phytochemicals in fermented soy products can be more efficacious when used together than when used individually. The benefits of using these complex fermented soy “foods” have historically been based on empirical observation, and are useful in embodiments of the present invention. These concepts help determine which combinations are truly likely to be efficacious.

Some of the mechanisms of how individual natural products, combinations of natural products, or various extracts of natural products help enhance our health and well being are not well understood. Various food supplements, nutrient formulations, spice and herbal formulations, and the like, that have biologically active ingredients have a variety of possible biochemical, nutritional and therapeutic functions. There are multiple factors arising from the organic constituents of higher plants and microorganisms that play an important role including carbohydrates, water-soluble organic acids, aromatic compounds, lipids, volatile alcohols, carbonyl compounds, terpenoids, steroids, flavonoids and isoflavonoids, amino acids & proteins, nucleic acids & derivatives, alkaloids, porphyrins, miscellaneous nitrogen and sulfur compounds, bacteriocins, phenolics, phospholipids, beta-glucans, and glutathione, etc. The literature suggests several situations in which cell viability (contained in ingested supplements, e.g., probiotics) is not required for some activities. Improved digestion of lactose, for example, in yogurt, fermented soy, and other modulation and anti-hypertensive effects, have been linked to nonviable cells such as cell components, enzymes activities, or fermentation products. It seems likely that the culturing in and of itself, apart from any vitamin or mineral enhancement, apart even from the presence of live active culture in the food, may create nutrients that are of extraordinary value in promoting health. One embodiment of the presently claimed invention provides fermented soy for use in combination with various natural products to provide health promoting compositions. In a principal embodiment, the fermented soy is used to grow certain mushroom species.

Preparation of Fermented Soybean Powder

One or more embodiments of the presently claimed invention include fermented soy as a base ingredient. The fermentation process appears to increase the bioavailability of the constituents in soy in part by removing the trypsin inhibitors found on the coating of soy that interfere with the absorption of nutritive compounds. Accordingly fermented soy may thereby offers a broader nutrient profile than non-fermented soy foods such as soy milk, soy nuts, tofu, and fresh or dry soybeans. Preferred fermented soy ingredients may be HAELAN 851®, HAELAN 951® (available from Haelan Products, Inc., Woodinville, Wash., USA), and SoyLac™ (available from FutureCeuticals, Momence, The HAELAN™ products, using low heat processing and other methodology, have been developed and improved over the last fifteen years by researchers in the United States and China. However, with over 10,000 varieties of soybeans and the extensive possible blending combinations of the isoflavones, protease inhibitors, saponins, phytosterols, inositol hexaphosphate compounds, phytates, phytic acid, and other beneficial compounds (including macronutrients and micronutrients), the HAELAN™ products are useful sources for preparation of the fermented soy powder ingredients of one or more embodiments in accordance with the claimed invention. In addition, the SoyLac products are also useful and serve as a next generation fermented soy product beyond HAELAN 951®. With knowledge of the extensive variation (i.e., up to 50%) in the nutritional value of soybeans based on the particular selected species, the geographical area reflecting soil chemistry, the age of the soybeans, as well as processing (handling, preparation, & fermentation), the HAELAN™ products were developed. The HAELAN 951® product is conveniently dehydrated to produce a dry powder used in the preparation of compositions of the invention. It is desirable to dehydrate or dry the soy fermentation product at as low a temperature as possible in order to avoid possible temperature degradation or reaction of ingredients. A representative dehydration process is described in detail in Example 1 below. Although HAELAN 851® and 951® are preferred sources for the preparation of powder soy with HAELAN 951® especially preferred, alternatively, any comparable microbial fermented soy preparation may be used and conveniently dehydrated to a powder, especially Stenotrophomonas spp. fermented product. HAELAN 951® contains the following isoflavones: daidzin, glycithin, genistin, acetyl glycitin, daidzein, glycitein, acetyl genistin, and genistein. HAELAN™ Products, Inc., Woodinville, Wash., 98072, U.S.A., has commercialized HAELAN 951® and other similar products in the United States as nutritional products. However, various health claims are made for these products in foreign countries. Research indicates that the benefits attributed to such fermented soybean products may be directly related to the soybean nutrients genistein, daidzein, protease inhibitors, saponins, phytosterols, phytic acid and other soybean compounds. The weight % of powdered fermented soy of the powder compositions of the invention, based on total weight of the composition, ranges in some formulations from about 0.5% to about 99.9% (i.e., soy+curcumin) and preferably about 50% in other formulations (i.e., soy, curcumin & other ingredients).

In the one or more embodiments of the presently claimed invention, any combination of spices, herbs, and natural products may be conveniently added to fermented soy powder to provide a pleasant taste, composition solubility, composition stability, and/or additional nutritional benefits. Spice ingredients include, but are not limited to, the following Indian spices that are potential sources of NF-kB blockers: turmeric (Curcuma longa), cumin (Cuminum cyminum), black pepper (Piper nigrum), cinnamon (Cinnamonum zeylanicum), coriander (Coriandrum sativum), red pepper (Capsicum frutescens), clove (Syzygium aromaticum), ginger (Zingiber officinale), tejpat (Cinnamon tamala), fennel (Foeniculum vulgare), Kaala Jeera (black cumin), cardamom (Amomum subulatum), kalonji (Nigella sativa), ajowan (Trachyspermum ammi), mint (Mentha piperta), sesame seeds (Sesamum indicum), asafetida (Ferula narthex), curry leaves (Murraya koenigil), pomergrate (Punica granatum), nutmeg (Myristicafragrans), saffron (Coccus sativus), dill (Anethum graveolens), oregano (Origanum vulgare), coriander (Coriandrum sativum), parsley (Petroselinum crispum), camphor (Cinnamomum camphor), licorice (Glycyrrhiza glabra), caraway (Carum carvl), mustard (Brassicajuncea), tamarind (Tamarindus indica), rosemary (Rosmarinus officinalis), onion (Alliumfistulosum), garlic (Allium sativum), almonds (Amygdalus comunis), cashews (Anacardium occidentale), walnut (Juglans nigra), mace (Myristica fragrans), mango (Magnifera indica), prunes (Prunus domestica), raisins (Vitis vinifera), pecans (Carya illinoensis), figs (Ficus carica), fenugreek (Trigonellafoneum-gracium), fenugreek seeds, ashwagandha, flax (Linum usitatissimum), til (Sesamum indicum), tulsi (Ocimum basilicum), and lemon grass (Cymbogogon filexusosus). Extracts, plant materials (e.g., ginseng), teas (e.g., tea, green tea, herbal teas, etc.), purified natural ingredients of plant materials or synthetic equivalents (e.g., vanilla), inorganic salts, and sweeteners individually or in any combination may also be added to the fermented soy-based nutritional formulations of the invention. Sweeteners can include either artificial sweeteners (e.g., cyclamate, saccharine, aspartame, acesulfame potassium, sucralose, sugar alcohols) or natural product sweeteners (stevioside, Stevia, sucrose, xylitol, Luohan, glucose, maltose, honey, etc.) may be added. Cocoa and chocolate powders are preferred additives. Lipids (e.g., lecithin, soy lecithin) may be added (e.g., as emulsifiers), carbohydrates (e.g., sugars, starches, etc.), grains, and malts (e.g., barley malt) may also be used as ingredients. Proteins may be added or the proteins from high protein content ingredients such as peanuts or soy may be used to provide a protein content of at least about 40% by weight, or more, based on a total weight of the particular embodiment of interest.

In powder form compositions of the invention, it is preferred to have: fermented soy; curcumin (Curcumin C3 Complex.®, available from Sabinsa Corp., East Windsor, N.J.), sucralose, and Stevia, and optionally to add other spices and herbs for specific nutritional needs, tastes, or product formulation requirements. Based on the total weight of the composition, it is preferred that powdered fermented soybean be manufactured from organically grown soybeans and comprise about 50% of compositions. Fermented soybean powder may range from about 20% to about 80% of the total weight of compositions. Curcumin is used preferably in an amount of about 3% (as Curcumin C3 Complex®), but may range from about 0.5% to about 6% based on the total weight of the composition. The equivalent amount of curcumin may be obtained from turmeric or other commercial curcumin products, but Curcumin C3 Complex® is especially preferred. Sucralose is used preferably in an amount of about 0.2%, but may range from about 0.1% to about 2.0% of the total weight of the composition. Stevia is used preferably in an amount of about 0.5%, but may range from about 0.1% to about 2.0% of the total weight of the composition. In powder formulations, optionally, in addition to fermented soy; curcumin (Curcumin C3 Complex®), sucralose, and Stevia ingredients, about 30-70%, preferably about 50%, (based on the total weight of the composition), of the ingredients of compositions of the invention are additional spices and herbs, especially spices and herbs earlier identified that are potential sources of NF-kB blockers.

The embodiments of powder compositions of the invention can be formulated in any suitable product form. Such product forms include, but are not limited to liquid, aerosol spray, cream, dispersion, emulsion, foam, gel, lotion, ointment, powder, solid and solution. The present compositions preferably include a vehicle. A useful vehicle is one that is medically acceptable for ingestion or topical application. Useful vehicles may include, but are not limited to, one or more aqueous systems, glycerin, alcohols, fatty alcohols, fatty ethers, fatty esters, polyols, glycols, vegetable oils, mineral oils, water, fruit juices, cultured dairy products, (e.g., yogurt), dairy products (e.g., milk), carbonated or non-carbonated beverages, and mixtures of the aforesaid. The powder compositions of the invention may be conveniently incorporated into a variety of liquids or solid compositions including, but not limited to, powders and other solids, liquid beverages (teas, mineral water, milk and other dairy products), capsules, tablets, ointments, skin creams, personal care products, pastas, soups, nutrient bars, cookies and other bakery items, etc.

A non-limiting embodiment of a powder formulation is described in Example 1 below when combined with mushroom species grown in fermented soy, or when combined with mushroom species grown in fermented soy with rice or rice flour, augmenting the growth medium of the fermented soy. The fermentation of the mushroom species is typically carried out with certain mushroom species and with certain prebiotic or probiotic bacterial species.

The nutraceutical compositions of the present invention may be administered in combination with a nutraceutically acceptable carrier. The active ingredients in such formulations may comprise from 1% by weight to 99% by weight, or alternatively, 0.1% by weight to 99.9% by weight. “Nutraceutically acceptable carrier” means any carrier, diluent or excipient that is compatible with the other ingredients of the formulation and not deleterious to the user.

The pharmaceutical compositions of the present invention may be administered in combination with a pharmaceutically acceptable carrier. The active ingredients in such formulations may comprise from 1% by weight to 99% by weight, or alternatively, 0.1% by weight to 99.9% by weight. “Pharmaceutically acceptable carrier” means any carrier, diluent or excipient that is compatible with the other ingredients of the formulation and not deleterious to the user.

The embodiments of the present invention include mushroom ingredients and components. Exemplary mushroom varieties include: Agaricus blazei (Brazilian Agaricus) Gilled Flamulina velutipes (Enokitake), Lentinula edodes (Shiitake), Pleurotus ostreatus (Oyster), Cordyceps sinensis (Caterpillar Mushroom), Entomogenous Auricularia auricula (Wood Ear), Tremella fuciformis (White Wood Ear), Polyporus umbellatus (Zhu-ling), Coriolus (Trametes) versicolor (Turkey tail), Grifola frondosa (Maitake), Ganoderma lucidum (Reishi), Inonotus obliquus (Chaga), Schizophylum commune (Split-gills), Hericium erinaceus (Monkey Head, Bear's Head), Poria cocos (Fu-ling), Phellinus igniarius (Tinder Box Fungus), Agrocybe cylindracea (Chestnut mushroom). Other useful mushroom species include A. bisporus, P. ostreatus, L. edodes, Auricularia auricula, Flammulina Velutipes, Volvariella volvacea. Agrocybe aegerita, Ganoderma spp., Grifolafrondosa, Hericium erinaceus, Hypsizygus marmoreus, Lepista nuda, Coprinus comatus, Pholiota nameko, and Stropharia spp.

In a further detailed description, examples of usable mushrooms include all of edible and medicinal mushrooms, e.g. Agaricus blazei, Ganderma lucidum, Grifola frondosa, Elfvingia applanata, Pleurotus ostreatus, Agaricus bisporus, Flammulina velutipes, Lentinus edodes and Crdyceps spp., but are not limited to them. Representative usable parts of the usable mushrooms are fruit bodies and mycelia. In the case of Agaricus blazei, Ganoderma lucidum, Grifola frondosa and Elfvingia applanata, the mushroom ingredients are obtained from their fruit (fruiting) bodies and mycelia are used. And in case of Pleurotus osteratus, Agaricus bisporus, Flammulina velutipes, Lentinus edodes and Cordyceps spp., the mushroom ingredients are obtained from their fruit bodies. Also, a mushroom ingredient can be a mixture of powder and extract from a mushroom. For example, a mushroom ingredient for dropping the blood sugar level is preferably an extract from Ganoderma lucidum.

Lactic Acid Fermented Solution of Cooked Soybeans, Rice and Mushroom

Lactic acid bacterium is a bacterium that produces lactic acid using a carbohydrate such as glucose and lactose, and has been used in fermented milk and cheese since about 3,000 B.C. Because of the fact that the milk fermented by lactic acid bacteria inhibits the growth of deleterious bacteria in digestive organs, and may prevent or delay some of the adverse effects of human aging, lactic acid bacteria fermented milk has been sold in goods worldwide to date. Many beneficial effects of lactic acid bacteria to human health have been studied broadly and comprehensively.

Some beneficial effects of lactic acid bacteria in an individual include: intestinal regulation (including prevention against diarrhea and constipation), suppression against the proliferation of intestinal cancers and aging by inhibiting the growth of deleterious bacteria, promotion of the growth by the formation of vitamins, prevention of adult diseases by controlling cholesterol, and reinforcement of immunity.

Typical examples of lactic acid bacteria include Streptococcus, Pediococcus, Leuconostoc, lactic acid bacillus, vipidus, etc. Lactic acid bacteria are found in the natural world in flora and fauna, e.g., digestive tracts of humans and animals and almost all of the vegetables. Bulgaricus bacteria, yogurt bacteria and thermophilus bacteria have been used in the production of yogurt. Yogurt bacteria, casei bacteria and acidophilus bacteria used in the production of beverages containing lactic acid bacteria. Casei bacteria and milk Streptococcus has been used in the production of cheese. Milk Streptococcus has been used in the production of fermented butter. Each specific lactic acid bacterium has been used in the manufacturing processes of different kinds of food.

Lactic acid bacteria live in the intestine or gut. Lactic acid bacteria secrete lactic acid, (lower) fatty acids, bacteriocin, H₂, O₂, etc, so as to inhibit the growth of deleterious bacteria and to lower the formation of cholesterol by HMG (Hydroxy Methyl Glutaric), Orotic Acid, Uric Acid, etc, which are formed by the fermentation of lactic acid bacteria. Particularly, Lactobacillus acidophilous directly decomposes cholesterol. Lactic acid bacteria activate the microphage detecting bacillus in immune systems, thereby detecting the appearance of bacteria and viruses, inhibiting the proliferation of cancer cell due to lymphocytic division, increasing the production of IgA, an antibody in blood, and promoting the production of .gamma.-interferon. Such a series of functions improves the immunity, increases nutritional values of food, inhibits endogenous infection, inhibits the formation of intestinal carcinogens, and results in the death of deleterious bacteria. The lactic acid bacteria as discussed above can be added to the fermented soy preparations described below in Examples 1, 2A and 2B.

For the purposes of this disclosure, the terms Dietary Reference Intakes (DRIB) and Recommended Dietary Allowances (RDAs) are as defined by the standards of the Food and Nutrition Board, Institute of Medicine, National Academy of Sciences, USA.

The methods described above may be further understood in connection with the following Examples. The following examples are provided for illustrative purposes and are not intended in and of themselves apart from the appended claims to be considered any more than non-limiting illustrative embodiments of the presently claimed invention.

### Example 1

### Preparation of Fermented Soy-Based Composition

Liquid HAELAN 951® is dehydrated in a two step process thus providing a dry fermented soy ingredient. Organically grown soybeans are used to produce the aqueous HAELAN 951® material. In the dehydration process, HAELAN 951® is first heated under vacuum to remove up to 80% of the water. The concentrated liquid then, under pressure, is passed through a tube the end of which has multiple small holes (about 0.2-0.5 mm d.). The concentrated liquid sprays out in a fog like state, and the tube rests on top of a large container with hot air blowing over the top. When the fog-like liquid encounters the hot air, the remaining water of the concentrate will be vaporized. The dry fermented soy powder drops down into the container and is collected.

Commercial powder forms of the following ingredients were added and thoroughly mixed with the fermented soy powder: Curcumin C3 Complex®, BIOPERINE® (black pepper extract), Piper longum, ginger, cardamom long, cinnamon, Barley malt, soy lecithin, cocoa, chocolate powder, sucralose, and stevia. Curcumin C3 Complex® contains three main chemical compounds: curcumin, demehtoxycurcumin and bisdemethoxycurcumin that are collectively known as curcuminoids. BIOPERINE® (available from Sabinsa Corp., East Windsor, N.J.) is an extract from the fruit of black pepper (Piper nigrum) or long pepper (Piper longum) that contains 95% of piperine (i.e., ca. 95% purity). The concentrations of the various ingredients are listed in Table 1 below.

TABLE 1
TABLE-US-00001 Ingredient Wt. % Weight (mg) Fermented Soy 50.00 10,000
Curcumin C3 Complex ® 2.50 500 BIOPERINE ® 0.03 5 Piper longum 0.10 20 Ginger 0.10 20
Cardamom long 0.10 20 Cinnamon 1.50 300 Barley malt 17.48 3,495 Soy lecithin 15.00 3,000
Cocoa 5.00 1,000 Chocolate powder 7.50 1,500 Sucralose* 0.20 40 Stevia** 0.50 100 TOTALS
100% 20,000 The color of the final composition is a light brown.
Possible Ingredient Ranges:
*10-150 mg
**10-100 mg

It is also contemplated that a mushroom as described above can be added to the final composition.

### Example 2A

### Preparation of Pleurotus Mushroom Grown in Fermented Soy

In accordance with an embodiment, rice flour (10% by weight based on the total mixture of the first two components) is added to the fermenting mixture of fermented soy-based composition of Example 1, and thoroughly mixed, then water added to provide a moisture content of about 60% by weight. The resulting substrate mixture is compacted into polypropylene bags to provide about 1-3 kg substrate per bag. The filled bags are stacked on racks and sterilized in an industrial autoclave for 2 hours at 121° C. The sterilized bags containing the fermented soy and rice growth medium are inoculated with Pleurotus spp. (e.g. Pleurotus ostreatus) fungal spawn. After a 20 to 25 day spawn run, the bags are removed and the substrate blocks (logs) are exposed to an environment conducive for browning of the exterior log surfaces. As the browning process reaches completion (4 weeks), primordia begin to form about 2 mm under the surface of the bag-log indicating that it is ready to produce mushrooms. Primordia maturation is stimulated by soaking the substrate in water (12° C.) for 3 to 4 hours at atmospheric pressure. Approximately 9 to 11 days after soaking, mushrooms are ready to harvest.

### Example 2B

### Preparation of Lentinula edodes Mushroom Grown in Fermented Soy

In accordance with an embodiment, rice flour (40% by weight based on the total mixture of the first two components) is added to the fermenting mixture of fermented soy-based composition of Example 1, and thoroughly mixed, then water added to provide a moisture content of about 60% by weight. The resulting substrate mixture is compacted into polypropylene bags to provide about 1-3 kg substrate per bag. The filled bags are stacked on racks and sterilized in an industrial autoclave for 2 hours at 121° C. The sterilized bags containing the fermented soy and rice growth medium are inoculated with Lentinula edodes fungal spawn. After a 20 to 25 day spawn run, the bags are removed and the substrate blocks (logs) are exposed to an environment conducive for browning of the exterior log surfaces. As the browning process reaches completion (4 weeks), primordia begin to form about 2 mm under the surface of the bag-log indicating that it is ready to produce mushrooms. Primordia maturation is stimulated by soaking the substrate in water (12° C.) for 3 to 4 hours at atmospheric pressure. Approximately 9 to 11 days after soaking, mushrooms are ready to harvest.

Alternatives and options regarding Examples 2A and 2B are as follows. It is further contemplated that in some embodiments the rice or rice flour can be omitted. Mushroom mycelia or basidiomycetes (selected from one or more mushrooms listed above) are used in the preparations. For example, in an alternative embodiment, the mushrooms can be grown over a period of 2-14 days in a controlled environment of temperature and humidity using the fermented soy growth medium having mushrooms according to Examples 1, 2A and 2B.

Further alternatives and options regarding Examples 2A and 2B are discussed in the following paragraphs. Substrates used in mushroom cultivation include both field-based residues and processing based-residues. However, as the nutrient composition of the substrate is one of the factors limiting colonization as well as quantitative and qualitative yield of cultivated mushrooms, supplements containing sugars and starch (easily available carbohydrates) and fats (slower degraded and time-lasting nutrient sources) are added to the basal ingredient. Supplements are used to increase nutritional content, speed-up growth and increase mushroom yield, especially in the cultivation of the white-rot mushroom fungi L. edodes and Pleurotus spp. The various organic supplements used in mushroom cultivation comprise molasses, brewer's grain, grasses and waste paper, cotton and coffee wastes etc. However, soybeans and cereal grains or their milling by-products are the most commonly used supplements, as they are generated in considerable amounts and contain increased levels of protein, fats and easily metabolized carbohydrates: soybeans (carbohydrates 21.5%, nitrogen (N) 6.3%), wheat bran (carbohydrates 49.8%, N 2.4%), rice bran (carbohydrates 37.0%, N 2.0%), rice flour, and millet (carbohydrates 57.3%, N1.9%).

Other agricultural wastes that can be used as substrates (alone or in combination with other supplements) in L. edodes cultivation are cereal straw, corn cobs, sugarcane bagasse, tea waste, sunflower seed hulls, peanut shells, vineyard prunings, cotton straw and seed hulls etc. The principle of the cultivation method comprises mixing and compacting ingredients into plastic bags, followed by sterilization, inoculation with fungal mycelia, incubation in dark rooms with controlled temperature and humidity for 30 to 80 days and finally fruiting induced by temperature reduction. Regardless of the main ingredients used, starch-based supplements such as wheat bran, rice bran, rice flour, millet, rye, corn, etc. are added to the mixture in a 10 to 40% ratio to the main ingredient. These supplements serve as nutrients to provide an optimum growing medium. Substrate ingredients are mixed, watered to gain a moisture content around 60% and filled into polypropylene bags to provide about 1-3 kg/bag. The filled bags are stacked on racks, loaded into an industrial-sized autoclave, sterilized for 2 hours at 121° C., cooled, and inoculated with spawn. After a 20 to 25 days spawn run, the bags are removed and the substrate blocks are exposed to an environment conducive for browning of the exterior log surfaces.

As the browning process reaches completion (4 weeks), primordia begin to form about 2 mm under the surface of the bag-log indicating that it is ready to produce mushrooms. Primordia maturation is stimulated by soaking the substrate in water (12° C.) for 3 to 4 hours at atmospheric pressure (or 3 to 4 min if vacuum soaking is used). Soaking allows water rapidly to displace carbon dioxide contained in air spaces, providing enough moisture for one flush of mushrooms. Approximately 9 to 11 days after soaking, mushrooms are ready to harvest.

This method decreases the production time and increases productivity. While in the traditional cultivation the logs need 8 months to 1 year of cultivation to produce 10-15 kg/100 kg of substrate, the cultivation on agro-forestry residues can furnish a yield of 60-80 kg/100 kg of substrate in 80 days harvest period. Two particular basidiomycetes mushroom genera that have received considerable attention for their nutritional value, medicinal properties and biodegradation abilities are Pleurotus and Lentinula. These widely cultivated edible mushrooms are efficient colonizers and bioconverters of lignocellulosic agroindustrial residues into palatable human food with medicinal properties. The efficacy of this value-added bioconversion process and the productivity of the mushroom crop are assessed by the biological efficiency (BE). Biological efficiency expresses the bioconversion of dry substrate to fresh fruiting bodies and indicates the fructification ability of the fungus utilizing the substrate. BE is calculated as the percentage ratio of the fresh weight of harvested mushrooms over the weight of dry substrate at inoculation. Yet, in a scarce number of reports, biological efficiency has been defined in terms of dry fruit bodies yield over the dry weight of the substrate used. Nevertheless, it should be made clear that apart from the type of substrate and stain used, yield response is determined by the duration of the cropping period and cultivation practice applied e.g. high spawn levels enhance mushroom yields. Nevertheless, in terms of Pleurotus cultivation, BE value should be over 50% to ensure profitability.

The genus Pleurotus comprises some the most popular edible mushrooms due to their favourable organoleptic and medicinal properties, fast mycelial growth and undemanding cultivation conditions. These mushrooms are commercially grown on pasteurized straw-based substrates or hardwood sawdust, fermented or not, with added supplements. However, as these fast-growing mushrooms display a complete lignocellulolytic enzyme system, they can use a wide spectrum of agricultural and industrial wastes that contain lignin and cellulose for growth and fruiting. A significant number of agroindustrial lignocellulosic materials are used as substrates for the production of Pleurotus spp., like corn cobs, various grasses and leaves, reed stems, maize and sorghum stover, rice and wheat straw, vine shoots, cardboard and paper, wood sawdust and chips, coffee pulp, cottonseed hulls, peanut shells, sunflower seed hulls, sugarcane and tequila bagasse, for example.

Further evaluation of the overall BE values obtained on these residue-substrates for P. ostreatus and P. pulmonarius strains may indicate that among all residues cardboard, coffee pulp, paper wastes and softwood residues, present the highest (>100%) biological efficiencies. BEs between 75% and 100% can be recorded on cotton wastes and wheat straw. If straw is pre-treated, an approximate 20% reduction of overall BE can be demonstrated when P. ostreatus is cultivated on non fermented wheat straw, as compared to fermented substrate.

On pretreated wheat straw, supplementation with cotton seed cake and soybean cake can enhance productivity of P. ostreatus. Finally, satisfactory productivity (BEs 50-75%) is demonstrated by most of agro-industrial residues, namely corncobs, various grasses and reed stems, vine shoots, cottonseed hulls and sugarcane bagasse. It is expected that P. pulmonarius can furnish significantly better yields than P. ostreatus on cardboard and soft-wood residues. Moreover, good yields of P. pulmonarius are expected on coffee and cotton wastes as well as on wheat straw. (See, Philippoussis, Antonios N., “Production of Mushrooms Using Agro-Industrial Residues as Substrates”, Biotechnology for Agro-Industrial Residues Utilisation, ed. Singh, P. & Pandey, A., ISBN: 978-1-4020-9941-0, pp. 163-96, 2009, herein incorporated by reference in its entirety.)

Examples of various other ingredients that may be included are those which are suitable for use in connection with the presently claimed invention with utility for various purposes by human and/or nonhuman subjects. With respect to a myriad of excipients, carriers, and other constituent ingredients that may be added to one or more embodiments described herein, one can readily refer to Joseph Price Remington et al., Remington's Pharmaceutical Sciences (17th ed. 1984-1985, Mack Publ., Easton, Pa.) and/or the USP National Formulary (USPNF) and other references and textbooks reciting such additional ingredients.

Various embodiments of the presently claimed or described invention(s) are described herein. However, it will be apparent by one of ordinary skill in the art that the presently claimed or described invention(s) may include variants and/or equivalents of the formulations or embodiments as described herein and still be within the scope of the presently claimed or described invention(s).

The following embodiments include one or more mushroom species grown in fermented soy (with or without rice flour) as the growth medium. Further probiotics described in the embodiments or claims may be those that produce lactic acid.

### EMBODIMENTS

### First Set

### Embodiment 1

A dietary supplement composition comprising: fermented soy; and curcumin, in amounts effective to provide nutritional benefits.

### Embodiment 2

The composition of embodiment 1, wherein the fermented soy is in an amount from about 80 wt. % to about 99.5 wt. % and the curcumin is in an amount from about 0.5 wt. % to about 20 wt. % based on a total weight of the composition.

### Embodiment 3

The composition of embodiment 1, wherein said curcumin further comprises demethoxycurcumin and bis-demethoxycurcumin.

### Embodiment 4

The composition of embodiment 1, wherein said fermented soy comprises fermented soy from Stenotrophomonas or fermented soy from Stenotrophomonas maltophilia.

### Embodiment 5

The composition of embodiment 1, further comprising at least one sweetener ingredient selected from the group consisting of cyclamate, saccharine, aspartame, sulfame potassium, sucralose, sugar alcohols, stevioside, stevia, sucrose, glucose, maltose and honey in an amount effective to provide a modified taste to the composition, wherein said modified taste is optionally a sweet taste.

### Embodiment 6

The composition of embodiment 5, wherein said at least one sweetener is in an amount from about 0.5 wt. % to about 2.0 wt. % based on the total weight of the composition.

### Embodiment 7

The composition of embodiment 6, wherein said sweetener ingredient is sucralose.

### Embodiment 8

The composition of embodiment 6, wherein said sweetener ingredient is stevia.

### Embodiment 9

The composition of embodiment 6, wherein said sweetener ingredient is sucralose and stevia.

### Embodiment 10

The composition of embodiment 1, further comprising at least one other plant ingredient or plant part thereof or extract thereof selected from the group consisting of: turmeric (Curcuma longa), cumin (Cuminum cyminum), black pepper, long pepper, (i) Piper nigrum, a Piper nigrum extract, Piper nigrum and its extract, or a high piperine content Piper nigrum extract with a piperine content of 95 wt. % of the high piperine content Piper nigrum extract, (ii) Piper longum, a Piper longum extract, Piper longum and its extract, or a high piperine content Piper longum extract with a piperine content of 95 wt. % of the high piperine content Piper longum extract, (iii) Piper longum/Piper nigrum, a Piper longum/Piper nigrum extract, Piper longum/Piper nigrum and their extract, or a high piperine content Piper longum/Piper nigrum extract with a piperine content of 95 wt. % of the high piperine content Piper longum/Piper nigrum extract, cinnamon (Cinnamonum zeylanicum), coriander (Coriandrum sativum), red pepper (Capsicum jrutescens), clove (Syzygium aromaticum), ginger (Zingiber officinale), tejpat (Cinnamon tamala), fennel (Foeniculum vulgare), Kaala Jeera (black cumin), cardamom (Amomum subulatum), cardamom long, kalonji (Nigella sativa), ajowan (Trachyspermum ammi), mint (Mentha piperta), sesame seeds (Sesamum indicum), asafetida (Ferula narthex), curry leaves (Murraya koenigii), pomergrate (Punica granatum), nutmeg (Myristicafragrans), saffron (Coccus sativus), dill (Anethum graveolens), oregano (Origanum vulgare), coriander (Coriandrum sativum), parsley (Petroselinum crispum), camphor (Cinnamomum camphor), licorice (Glycyrrhiza glabra), caraway (Carum carvi), mustard (Brassicajuncea), tamarind (Tamarindus indica), rosemary (Rosmarinus officinalis), onion (Alliumfistulosum), garlic (Allium sativum), almonds (Amygdalus comunis), cashews (Anacardium occidentale), walnut (Juglans nigra), mace (Myristicafragrans), mango (Magnifera indica), prunes (Prunus domestica), raisins (Vitis vinifera), pecans (Carya illinoensis), figs (Ficus carica), fenugreek (Trigonellafoneum-gracium), fenugreek seeds, flax (linum usitatissimum), til (Sesamum indicum), tulsi (Ocimum basilicum), lemon grass (Cymbogogon flexusosus), ginseng, green tea, lutein, ashwaghanda, caraway seed, juniper berries, spearmint, mace, fennel, nutmeg, sage, hyssop, bee balm, lemon balm, sage, savory, thyme, bilberry, St. John's Wort, milk thistle, tea, green tea, purslane, grape seed, ginko, King Solomon's Seal, peppermint, sassafras, cocoa, chocolate, lipids, lecithin, soy lecithin, carbohydrates, sugars, starches, grains, grain malts, and barley malt.

### Embodiment 11

The composition of embodiment 10, wherein said at least one other plant ingredient or plant part or extract thereof is in an amount from about 0.1 wt. % to about 20 wt. % based on the total weight of the composition.

### Embodiment 12

The composition of embodiment 1, further comprising: at least one sweetener ingredient selected from the group consisting of cyclamate, saccharine, aspartame, acesulfame potassium, sucralose, sugar alcohols, stevioside, stevia, sucrose, glucose, maltose and honey in an amount from about 0.5 wt. % to about 2.0 wt. % based on the total weight of the composition; at least one other plant ingredient or plant part or extract thereof selected from the group consisting of: turmeric (Curcuma longa), cumin (Cuminum cyminum), black pepper, long pepper, (i) Piper nigrum, a Piper nigrum extract, Piper nigrum and its extract, or a high piperine content Piper nigrum extract with a piperine content of 95 wt. % of the high piperine content Piper nigrum extract, (ii) Piper longum, a Piper longum extract, Piper longum and its extract, or a high piperine content Piper longum extract with a piperine content of 95 wt. % of the high piperine content Piper longum extract, (iii) Piper longum/Piper nigrum, a Piper longum/Piper nigrum extract, Piper longum/Piper nigrum and their extract, or a high piperine content Piper longum/Piper nigrum extract with a piperine content of 95 wt. % of the high piperine content Piper longum/Piper nigrum extract, cinnamon (Cinnamonum zeylanicum), coriander (Coriandrum sativum), red pepper (Capsicumfrutescens), clove (Syzygium aromaticum), ginger (Zingiber officinale), tejpat (Cinnamon tamala), fennel (Foeniculum vulgare), Kaala Jeera (black cumin), cardamom (Amomum subulatum), kalonji (Nigella sativa), ajowan (Trachyspermum ammi), mint (Mentha piperta), sesame seeds (Sesamum indicum), asafetida (Ferula narthex), curry leaves (Murraya koenigii), pomergrate (Punica granatum), nutmeg (Myristicafragrans), saffron (Coccus sativus), dill (Anethum graveolens), oregano (Origanum vulgare), coriander (Coriandrum sativum), parsley (Petroselinum crispum), camphor (Cinnamomum camphor), licorice (Glycyrrhiza glabra), caraway (Carum carvi), mustard (Brassicajuncea), tamarind (Tamarindus indica), rosemary (Rosmarinus officinalis), onion (Alliumfistulosum), garlic (Allium sativum), almonds (Amygdalus comunis), cashews (Anacardium occidentale), walnut (Juglans nigra), mace (Myristicafragrans), mango (Magnifera indica), prunes (Prunus domestica), raisins (Vitis vinifera), pecans (Carya illinoensis), figs (Ficus carica), fenugreek (Trigonellafoneum-gracium), fenugreek seeds, flax (linum usitatissimum), til (Sesamum indicum), tulsi (Ocimum basilicum), lemon grass (Cymbogogon flexusosus), ginseng, green tea, lutein, ashwaghanda, caraway seed, juniper berries, spearmint, mace, fennel, nutmeg, sage, hyssop, bee balm, lemon balm, sage, savory, thyme, bilberry, St. John's Wort, milk thistle, tea, green tea, purslane, grape seed, ginko, King Solomon's Seal, peppermint, sassafras, cocoa, chocolate powder, lipids, lecithin, soy lecithin, carbohydrates, sugars, starches, grains, grain malts, and barley malt, in an amount from about 0.1 wt. % to about 20 wt. %, based on the total weight of the composition

### Embodiment 13

The composition of embodiment 12, wherein said fermented soy comprises soy fermented with Stenotrophomonas or soy fermented with Stenotrophomonas maltophilia, and said fermented soy is optionally provided in liquid form, dehydrated form, powder form, or dehydrated powder form; wherein said curcumin further comprises demethoxycurcumin and bis-demethoxycurcumin; wherein said at least one sweetener ingredient is an admixture of stevia and sucralose; and wherein said at least one other ingredient, plant or plant part, or extract thereof is an admixture of: (i) Piper nigrum, a Piper nigrum extract, Piper nigrum and its extract, or a high piperine content Piper nigrum extract with a piperine content of 95 wt. % of the high piperine content Piper nigrum extract; (ii) Piper longum, a Piper longum extract, Piper longum and its extract, or a high piperine content Piper longum extract with a piperine content of 95 wt. % of the high piperine content Piper longum extract; (iii) Piper longum/Piper nigrum, a Piper longum/Piper nigrum extract, Piper longum/Piper nigrum and their extract, or a high piperine content Piper longum/Piper nigrum extract with a piperine content of 95 wt. % of the high piperine content Piper longum/Piper nigrum extract; (iv) ginger; (v) cardamom long; (vi) cinnamon; (vii) barley malt; (viii) soy lecithin; (ix) cocoa; and (x) chocolate powder.

### Embodiment 14

A dietary supplement composition comprising: fermented soy;
curcumin; at least one sweetener ingredient selected from the group consisting of cyclamate, saccharine, aspartame, acesulfame potassium, sucralose, sugar alcohols, stevioside, stevia, sucrose, glucose, maltose, xylitol, luohan and honey; at least one additional plant ingredient or plant part or extract thereof selected from the group consisting of: turmeric (Curcuma longa), cumin (Cuminum cyminum), black pepper, long pepper, (i) Piper nigrum, a Piper nigrum extract, Piper nigrum and its extract, or a high piperine content Piper nigrum extract with a piperine content of 95 wt. % of the high piperine content Piper nigrum extract, (ii) Piper longum, a Piper longum extract, Piper longum and its extract, or a high piperine content Piper longum extract with a piperine content of 95 wt. % of the high piperine content Piper longum extract, (iii) Piper longum/Piper nigrum, a Piper longum/Piper nigrum extract, Piper longum/Piper nigrum and their extract, or a high piperine content Piper longum/Piper nigrum extract with a piperine content of 95 wt. % of the high piperine content Piper longum/Piper nigrum extract, cinnamon (Cinnamonum zeylanicum), coriander (Coriandrum sativum), red pepper (Capsicum frutescens), clove (Syzygium aromaticum), ginger (Zingiber officinale), tejpat (Cinnamon tamala), fennel (Foeniculum vulgare), Kaala Jeera (black cumin), cardamom (Amomum subulatum), kalonji (Nigella sativa), ajowan (Trachyspermum ammi), mint (Mentha piperta), sesame seeds (Sesamum indicum), asafetida (Ferula narthex), curry leaves (Murraya koenigi/), pomergrate (Punica granatum), nutmeg (Myristicafragrans), saffron (Coccus sativus), dill (Anethum graveolens), oregano (Origanum vulgare), coriander (Coriandrum sativum), parsley (Petroselinum crispum), camphor (Cinnamomum camphor), licorice (Glycyrrhiza glabra), caraway (Carum carvi), mustard (Brassicajuncea), tamarind (Tamarindus indica), rosemary (Rosmarinus officinalis), onion (Alliumfistulosum), garlic (Allium sativum), almonds (Amygdalus comunis), cashews (Anacardium occidentale), walnut (Juglans nigra), mace (Myristicafragrans), mango (Magnifera indica), prunes (Prunus domestica), raisins (Vilis vinifera), pecans (Carya illinoensis), figs (Ficus carica), fenugreek (Trigonellafoneum-gracium), fenugreek seeds, flax (linum usitatissimum), til (Sesamum indicum), tulsi (Ocimum basilicum), lemon grass (Cymbogogon flexusosus), ginseng, green tea, lutein, ashwaghanda, caraway seed, juniper berries, spearmint, mace, fennel, nutmeg, sage, hyssop, bee balm, lemon balm, sage, savory, thyme, bilberry, St. John's Wort, milk thistle, tea, green tea, purslane, grape seed, ginko, King Solomon's Seal, peppermint, sassafras, cocoa, chocolate, lipids, lecithin, soy lecithin, carbohydrates, sugars, starches, grains, grain malts, and barley malt; and a pharmaceutically or cosmetically acceptable carrier.

### Embodiment 15

The composition of embodiment 14, wherein said carrier is selected from the group consisting of water, aqueous systems, tea, coffee, fruit juices, glycerin, alcohols, fatty alcohols, fatty ethers, fatty esters, polyols, glycols, vegetable oils, mineral oils, cultured dairy products, dairy products, carbonated and uncarbonated beverages.

### Embodiment 16

The composition of embodiment 14, wherein said fermented soy is in an amount from about 0.5 wt. % to about 90 wt. %, said curcumin is in an amount from about 0.5 wt. % to about 20 wt. %, said sweetener ingredient is in an amount from about 0.1 wt. % to about 2.0 wt. %, said at least one additional plant ingredient or plant part or extract thereof is in an amount from about 0.1 wt. % to about 20 wt. %, said carrier is in an amount from about 0.5 wt. % to about 90 wt. % based on the total weight of the composition.

### Embodiment 17

The composition of embodiment 16, wherein said composition is in a product form selected form the group consisting of liquid, aerosol spray, cream, emulsion, solid, dispersion, foam, gel, lotion, ointment, powder, solution, tablets, bakery items, and capsules.

### Embodiment 18

The composition of embodiment 14, wherein said fermented soy comprises from about 0.5 wt. % to about 90 wt. % of said composition, said fermented soy comprises soy fermented with Stenotrophomonas or soy fermented with Stenotrophomonas maltophilia, and said fermented soy is optionally provided in liquid form, dehydrated form, powder form, or dehydrated powder form; wherein said curcumin comprises from about 0.5 wt. % to about 20 wt. % of said composition, and said curcumin further comprises demethoxycurcumin and bis-demethoxycurcumin; wherein said at least one sweetener ingredient comprises from about 0.1 wt. % to about 2.0 wt. % of said composition and said at least one sweetener ingredient comprises stevia and sucralose; wherein said at least one additional plant ingredient or plant part or extract thereof comprises from about 0.1 wt. % to about 20 wt. % of said composition and said at least one additional plant ingredient or plant part or extract thereof comprises black pepper (Piper nigrum), long pepper (Piper longum), Piper nigrum extract, Piper longum extract, a high piperine content Piper nigrum extract with a piperine content of 95 wt. % of the high piperine content Piper nigrum extract, a high piperine content Piper longum extract with a piperine content of 95 wt. % of the high piperine content Piper longum extract, a high piperine content Piper longum/Piper nigrum extract with a piperine content of 95 wt. % of the high piperine content Piper longum/Piper nigrum extract, ginger, cardamom long, cinnamon, barley, malt, soy lecithin, cocoa, and chocolate; and wherein said carrier comprises from about 0.5 wt. % to about 90 wt. % of water, based on a total weight of the composition.

### Embodiment 19

The composition of embodiment 1, wherein said fermented soy is obtained by a method comprising the steps of heating liquid fermented soy under vacuum to remove water and to produce a concentrated fermented soy composition; vaporizing said concentrated fermented soy composition to form a vapor-treated soy composition; and removing additional water from the vapor-treated soy composition.

### Embodiment 20

The composition of embodiment 19, wherein said fermented soy comprises soy fermented with Stenotrophomonas or soy fermented with Stenotrophomonas maltophilia.

### Embodiment 21

The composition of embodiment 19, wherein said step of removing additional water comprises the step of removing water from said vapor-treated soy composition in an amount sufficient to produce a powder form of said composition.

### Embodiment 22

The composition of embodiment 19, wherein said step of vaporizing is conducted under pressure.

### Embodiment 23

The composition of embodiment 22, wherein said step of removing additional water further comprises exposing said vapor-treated soy composition to hot air sufficient to remove remaining water from said vapor-treated soy composition to form a powder.

### Embodiment 24

The composition of embodiment 13, comprising (ingredient/wt %/wt(mg)): Fermented Soy/50.00/10,000; Curcumin, demethoxycurcumin and bis-demethoxycurcumin/2.50/500; Extract of Piper nigrum/Piper longum with 95 wt. % piperine content based on the total weight of the extract/0.03/5; Piper longum/0.10/20; Ginger/0.10/20; Cardamom long/0.10/20; Cinnamon/1.50/300; barley malt/17.48/3495; soy lecithin/15.00/3000; cocoa/5.00/1000; chocolate powder/7.50/1500; sucralose/0.20/40; stevia/0.50/100; wherein an amount of said sucralose may be adjusted to range from about 10 mg to about 150 mg; and wherein an amount of said stevia may be adjusted to range from about 10 mg to about 100 mg.

### Embodiments

### Second Set

Additional embodiments that are illustrative and non-limiting are provided herein.

### Embodiment 1

A composition comprising: (a) a mushroom grown in a fermented soy growth medium; and (b) curcuminoids comprising one or more of curcumin, demethoxycurcumin and bis-demethoxycurcumin.

### Embodiment 2

The composition of embodiment 1, wherein the fermented soy growth medium comprises fermented soy.

### Embodiment 3

The composition of embodiment 2, wherein the fermented soy includes fermented soy fermented with one or more of: (c) Lactobacillus bulgaricus, (d) Lactobacillus casei, (e) Lactobacillus plantarum, (f) Streptococcus thermophilus, (g) Bifidobacterium bifidum, (h) Bifidobacterium breve, (i) Bifidobacterium infantis, (j) Bifidobacterium longum, (k) Bifidobacterium lactis, (l) Lactobacillus acidophilus, (m) Lactobacillus bulgaricus, (n) Lactobacillus casei, (o) Lactobacillus helveticus, (p) Lactobacillus plantarum, (q) Lactobacillus rhamnosus, (r) Lactobacillus salviarious, (s) Lactobacillus johnsonii, (t) Lactobacillus jermentum, (u) Lactobacillus lactis, (v) Lactobacillus sanjranciscensis, (w) Pediococcus acidilactici, (x) Streptococcus jaccium, (y) B. Streptococcus thermophilus, (z) a probiotic bacteria, and (aa) a prebiotic bacteria.

### Embodiment 4

The composition of embodiment 2, wherein the fermented soy growth medium further comprises rice flour.

### Embodiment 5

The composition of embodiment 1, wherein the mushroom is a dried mushroom, a spray-dried mushroom, a drum-dried mushroom, or combinations thereof.

### Embodiment 6

The composition of embodiment 1, wherein the mushroom is a powdered mushroom.

### Embodiment 7

The composition of embodiment 1 further comprising piperine from Piper nigrum, Piper longum or a combination thereof.

### Embodiment 8

The composition of embodiment 4 further comprising at least 95% piperine, i.e. having a purity of 95%.

### Embodiment 9

The composition of embodiment 5, wherein the mushroom species is one or more of: (bb) Agaricus blazei, (cc) Flamulina velutipes, (dd) Lentinula edodes, (ee) Pleurotus ostreatus, (ff) Cordyceps sinensis, (gg) Auricularia auricula, (hh) Tremella focilormis, (ii) Polyporus umbellatus, (jj) Coriolus versicolor, (kk) Grifola frondosa, (ll) Ganoderma lucidum, (mm) Inonotus obliquus, (nn) Schizo phylum commune, (oo) Hericium erinaceus, (pp) Poria cocos, (qq) Phellinus igniarius, (a) Agrocybe cylindracea, (ss) A. bisporus, (tt) Volvariella volvacea, (uu) Agrocybe aegerita, (vv) Ganoderma spp., (ww) Hypsizygus marmoreus, (xx) Lepista nuda, (yy) Coprinus comatus, (zz) Pholiota nameko, (aaa) Stropharia spp., (bbb) Elfvingia applanata, and (ccc) Cordyceps spp.

### Embodiment 10

The composition of embodiment 1, wherein the fermented soy is fermented with one or more of: (ddd) L. acidophilus, (eee) L. Bulgaricus, (fff) L. casei, (ggg) L. plantarum, and (hhh) S. thermophilus.

### Embodiment 11

The composition of embodiment 1 further comprising a member selected from the group consisting of: fiber, protein, fruit(s), vegetable(s), spice(s), botanical plant part(s), botanical plant extracts, tuberous material, vitamin(s), animal mineral(s), plant mineral(s), mineral(s), anti-oxidants(s), daidzein, genistein, daidzin, genistin, miso, natto, ocean herb(s), tea, green tea, coffee, cultured soy, lactic acid, turmeric, flavoring agent(s), sweetener(s), water, carbonated water, plain water, potable water, sparkling water, sparkling ginger, vanilla flavor, coffee flavor, antiemetic, coke syrup, soybean, black soybean, corn, blue corn, wild blueberry, acai, blackcurrant, sweet cherry, raspberry, elderberry, blackberry, aronia, wild bilberry, red wine powders and extracts, pomegranate, blackcurrants, cranberries, cherry powders and extracts, wild blueberry extract, wild blueberries, grapes, grape seed, raspberry seed, cranberries, prunes, cherries, strawberries, cranberry, grape seed extract, citric acid, mangosteen, acerola, camu camu, whole coffee fruit, coffee extracts and powders, mango, pineapple, noni powder, grapefruit, lemon, tomato, spinach, aloe vera, peas, alfalfa, garlic, nettles, ginger, spices, heart-healthy grain-based fibers, traditional ancient grains, adzuki, barley bran, oats, 55% oat-soluble freeze-dried fiber based on a weight of the fiber itself, beta-glucans, powdered sprout concentrate blends of broccoli, water cress, daikon radish, alfalfa, amaranth, cabbage, kale, mustard, onions, quinoa, wheat, flax seed, red clover and cauliflower sprouts, winter cherry, licorice, giant potato, white musale, indian gooseberry, aloeweed, elephant creeper, black musale, caper plant, indian gum arabic tree, amla, pedalium, indian timospora, cinnamon, nutmeg, cloves, aniseed, com, chromium beet, banana, freeze dried banana powder, drum dried banana powder, aronia, apple, orange, acerola, peanut, rice, carrot, sprouts, anthocyanin(s), antioxidant, antioxidant blend, beta carotene, boron, chromium, ellagic acid and ellagitannins, essential sugar(s), glucosinolate(s), isoflavone(s), lutein, lycopene, oligomeric proanthocyanidin(s), polyphenol(s), punicalagin(s), quercitin, quinic acid, reservatol, water soluble fiber, water soluble beta glucan(s), sulforaphane, vitamin C, ascorbic acid, essential monosaccharides(s), mannose, fucose, xylose, galactose, glucose, N-acetylneuraminic acid (sialic acid), N-acetylgalactosamine, N-acetylglucosamine, and combinations or any sub combinations of any of the above.

### Embodiment 12

The composition of embodiment 1 provided in one or more forms selected from the group consisting of: smoothie, soup, nutritional bar, snack, cereal, breads, cake, oatmeal cookie, cookie, dessert, spread, salad dressing, beverage, specialty beverage, carbonated beverage, energy beverage, functional beverage, energy beverage, diet beverage, soda, capsule, tablet, caplet, emulsion, suspension, syrup, liquid, solid dosage form, liquid dosage form, pet food, baked food, food, cooked food, cosmetic, nutritional supplement, shake, ice cream, yogurt, capsule, green drink, antioxidant beverage, weight management beverage, meal replacement beverage, lemonade, tropical beverage, electrolytic beverage, herbal tea beverage, tea, coffee, paste, meal replacement paste, meal replacement paste without water, meal replacement paste with at least 40% by weight protein, protein from peanuts, peanut oil, oil, canola oil, olive oil, and combinations or any sub-combinations thereof.

### Embodiment 13

The composition of embodiment 1 further comprising one or more powder(s) selected from the group consisting of: fruit powder, acai powder, freeze dried acai powder, freeze dried acai powder with 1% anthocyanin(s), organic freeze dried acai powder with 1% anthocyanin(s), freeze dried acai powder, drum dried acai powder, drum dried acai flakes, drum dried apple powder, freeze dried apricot powder, aronia and/or chokeberry with 1.5% anthocyanin(s) based on a total weight of the chokeberry, freeze dried aronia powder, freeze dried banana powder, bilberry with 1.5% anthocyanin(s) based on a total weight of the bilberry, bilberry plus 5% anthocyanin(s) based on the total weight of the bilberry, freeze dried bilberry powder, blackberry with 0.8% anthcyanins based on a total weight of the blackberry, freeze dried blackberry powder, freeze dried blackcurrant powder, freeze dried wild blueberry powder, drum dried blueberry powder, wild blueberry with 5% anthocyanin(s) based on a total weight of the wild blueberry, wild blueberry with 1000 ppm pterostilbene present in the wild blueberry, freeze dried wild blueberry powder, freeze dried blueberry powder, drum dried tart cherry with 0.05% anthocyanin(s) based on a total weight of the dried cherry, freeze dried tart cherry with 0.5% anthocyanin(s) based on the total weight of the dried cherry, freeze dried sweet cherry with 0.7% anthocyanin(s) based on the total weight of the dried cherry, drum dried sweet cherry with 0.05% anthocyanin(s) based on the total weight of the dried cherry, freeze dried sweet cherry, drum dried sweet cherry, chokeberry and/or aronia with 1.5% anthocyanin(s) based on a total weight of the aronia, cranberry with 4% quinic acid based on a total weight of the cranberry, cranberry with 6% quinic acid based on the total weight of the cranberry, drum dried cranberry powder, freeze dried cranberry powder, white cranberry with 3% quinic acid based on the total weight of the cranberry, elderberry with 4% anthocyanin(s) based on a total weight of the elderberry, goji and/or wolfberry powder 20% based on the total weight of the wolfberry, spray-dried concord grape, grape powder, grapefruit powder, kiwi powder, lemon powder, freeze dried lychee powder, mango powder, aid-dried mangosteen powder, freeze dried mangosteen powder, maqui powder, noni powder, granulated noni, orange segment powder, peach powder, pear powder, persimmon powder, pineapple powder, plum powder, pomegranate powder, pomegranate with 5% ellagic acid based on a total weight of the pomegranate, prune powder, raspberry with 0.7% anthocyanin(s) based on a total weight of the raspberry, black raspberry, raspberry seed powder, raspberry powder, drum dried seedless raspberry powder, strawberry powder with 5% phenolics based on a total weight of the strawberry, drum dried strawberry flakes, strawberry powder, strawberry seed powder, drum dried watermelon powder, freeze dried watermelon powder, wolfberry and/or goji powder 20% based on a total weight of the goji powder, yum berry, and combinations thereof or any sub-combinations thereof.

### Embodiment 14

The composition of embodiment 1 further comprising one or more ingredients selected from the group consisting of: ascorbic acid, ascorbic acid in crystalline form, ashwagandha, barley, barley malt, carotene, beta-carotene, bilberry, piperine, piperine 95%, and any combination or any sub-combination thereof.

### Embodiment 15

The composition of embodiment 14, in the form of a beverage wherein said one or more ingredients are provided in an amount sufficient to dissolve in a beverage stored at room temperature or stored from about 5 to about 15° C.

### Embodiment 16

The composition of anyone of embodiments 1-15 provided in a therapeutically effective amount for the treatment or symptomatic relief of a condition selected from the group consisting of: breast cancer, lung cancer, prostate cancer, leukemia, cancer, recurrence of cancer, cardiovascular disease, adult onset diabetes, Crohn's disease, irritable bowel syndrome, ulcerative colitis, skin rash, psoriasis, atopic dermatitis, arthritis, tumor metastasis or tumor growth, infection, rheumatism, diabetic wound, runny nose, cough, liver disorder, cirrhosis, precancerous cancer progression, jaundice, Alzheimer's disease, menopause, symptoms of menopause, an inflammatory condition, malnutrition in a child from an age of 1 up to and including 6 years, malnutrition in a child from an age between 6 up to and including 10 years, malnutrition in a child from an age between 10 up to and including 15 years, malnutrition in an adolescent child from an age between 15 up to and including 17 years, malnutrition in a baby suffering from HIV infection, malnutrition in a child suffering from HIV infection, malnutrition in a person or individual afflicted with HIV infection, malnutrition in a male individual, malnutrition in a female individual, malnutrition in a person from an age between about 17 up to and including 99 years, an HIV condition, an underweight condition, an immune compromised condition, a condition amenable to relief by ingestion of the composition(s), a condition amenable to symptomatic relief by ingestion of the composition(s) in a person in need thereof, a condition amenable to treatment or relief by ingestion of the composition(s) in a person in need thereof.

### Embodiment 17

The composition of anyone of embodiments 1-16, wherein if a vitamin is present in the composition, then it is present in an amount not exceeding its total recommended dietary allowance (RDA) unless safe to exceed its (RDA) for treating or providing symptomatic relief anyone of the conditions being treated or for which relief is sought in a person in need thereof.

### Embodiment 18

The composition of any one of embodiments 1-17, wherein the composition comprises vitamin E in an amount of about 1 IU, 2 IU, 3 IU, 4 IU, 5 IU, 6 IU, 7 IU, 8 IU, 9 IU, 10 IU, 15 IU, 20 IU, 25 IU, 30 IU, 35 IU, 40 IU, 45 IU, 50 IU, 55 IU, 60 IU, 65 IU, 70 IU, 75 IU, 80 IU and being within about at least one of or just one of ±

±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, and ±25% for each of the IV values, respectively.

### Embodiment 19

The composition of any one of embodiments 1-18, wherein the composition comprises vitamin C in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, and ±25% for each of the mg values, respectively.

### Embodiment 20

The composition of any one of embodiments 1-19, wherein the composition comprises vitamin B-1 in an amount of about 10 mcg, 15 mcg, 20 mcg, 25 mcg, 30 mcg, 35 mcg, 40 mcg, 45 mcg, 50 mcg, 55 mcg, 60 mcg, 65 mcg, 70 mcg, 75 mcg, 80 mcg, 85 mcg, 90 mcg, 95 mcg, 100 mcg, 105 mcg, 110 mcg, 115 mcg, 120 mcg, 125 mcg, 130 mcg, 135 mcg, 140 mcg, 145 mcg, 150 mcg, 160 mcg, 170 mcg, 180 mcg, 190 mcg, 200 mcg, 210 mcg, 220 mcg, 230 mcg, 240 mcg, 250 mcg, 260 mcg, 270 mcg, 280 mcg, 290 mcg, 300 mcg, 310 mcg, 320 mcg, 330 mcg, 340 mcg, 350 mcg and being within about ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, and ±25% for each of the mcg values, respectively.

### Embodiment 21

The composition of anyone of embodiments 1-20, wherein the composition comprises vitamin B-2 in an amount of about 10 mcg, 15 mcg, 20 mcg, 25 mcg, 30 mcg, 35 mcg, 40 mcg, 45 mcg, 50 mcg, 55 mcg, 60 mcg, 65 mcg, 70 mcg, 75 mcg, 80 mcg, 85 mcg, 90 mcg, 95 mcg, 100 mcg, 105 mcg, 110 mcg, 115 mcg, 120 mcg, 125 mcg, 130 mcg, 135 mcg, 140 mcg, 145 mcg, 150 mcg, 160 mcg, 170 mcg, 180 mcg, 190 mcg, 200 mcg, 210 mcg, 220 mcg, 230 mcg, 240 mcg, 250 mcg, 260 mcg, 270 mcg, 280 mcg, 290 mcg, 300 mcg, 310 mcg, 320 mcg, 330 mcg, 340 mcg, 350 mcg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, or ±25% for each of the mcg values, respectively.

### Embodiment 22

The composition of any one of embodiments 1-21, wherein the composition comprises vitamin B-3 in an amount of about 100 mcg, 150 mcg, 200 mcg, 250 mcg, 300 mcg, 350 mcg, 400 mcg, 450 mcg, 500 mcg, 550 mcg, 600 mcg, 650 mcg, 700 mcg, 750 mcg, 800 mcg, 850 mcg, 900 mcg, 950 mcg, 1000 mcg, 1050 mcg, 1100 mcg, 1150 mcg, 1200 mcg, 1250 mcg, 1300 mcg, 1350 mcg, 1400 mcg, 1450 mcg, 1500 mcg, 1600 mcg, 1700 mcg, 1800 mcg, 1900 mcg, 2000 mcg, 2100 mcg, 2200 mcg, 2300 mcg, 2400 mcg, 2500 mcg, 2600 mcg, 2700 mcg, 2800 mcg, 2900 mcg, 3000 mcg, 3100 mcg, 3200 mcg, 3300 mcg, 3400 mcg, 3500 mcg, 3600 mcg, 3700 mcg, 3800 mcg, 3900 mcg, 4000 mcg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, and ±25% for each of the mcg values, respectively.

### Embodiment 23

The composition of anyone of embodiments 1-22, wherein the composition comprises vitamin B-6 in an amount of about 10 mcg, 15 mcg, 20 mcg, 25 mcg, 30 mcg, 35 mcg, 40 mcg, 45 mcg, 50 mcg, 55 mcg, 60 mcg, 65 mcg, 70 mcg, 75 mcg, 80 mcg, 85 mcg, 90 mcg, 95 mcg, 100 mcg, 105 mcg, 110 mcg, 115 mcg, 120 mcg, 125 mcg, 130 mcg, 135 mcg, 140 mcg, 145 mcg, 150 mcg, 160 mcg, 170 mcg, 180 mcg, 190 mcg, 200 mcg, 210 mcg, 220 mcg, 230 mcg, 240 mcg, 250 mcg, 260 mcg, 270 mcg, 280 mcg, 290 mcg, 300 mcg, 310 mcg, 320 mcg, 330 mcg, 340 mcg, 350 mcg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, or ±25% for each of the mcg values, respectively.

### Embodiment 24

The composition of anyone of embodiments 1-23, wherein the composition comprises folate in an amount of about 10 mcg, 15 mcg, 20 mcg, 25 mcg, 30 mcg, 35 mcg, 40 mcg, 45 mcg, 50 mcg, 55 mcg, 60 mcg, 65 mcg, 70 mcg, 75 mcg, 80 mcg, 85 mcg, 90 mcg, 95 mcg, 100 mcg, 105 mcg, 110 mcg, 115 mcg, 120 mcg, 125 mcg, 130 mcg, 135 mcg, 140 mcg, 145 mcg, 150 mcg, 160 mcg, 170 mcg, 180 mcg, 190 mcg, 200 mcg, 210 mcg, 220 mcg, 230 mcg, 240 mcg, 250 mcg, 260 mcg, 270 mcg, 280 mcg, 290 mcg, 300 mcg, 310 mcg, 320 mcg, 330 mcg, 340 mcg, 350 mcg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, or ±25% for each of the mcg values, respectively.

### Embodiment 25

The composition of anyone of embodiments 1-24, wherein the composition comprises calcium in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, and ±25% for each of the mg values, respectively.

### Embodiment 26

The composition of anyone of embodiments 1-25, wherein the composition comprises phosphorus in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, and ±25% for each of the mg values, respectively.

### Embodiment 27

The composition of any one of embodiments 1-26, wherein the composition comprises potassium in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, 1000 mg, 1100 mg, 1200 mg, 1200 mg, 1300 mg, 1400 mg, 1500 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, and ±25% for each of the mg values, respectively.

### Embodiment 28

The composition of anyone of embodiments 1-27, wherein the composition comprises fermented soy in an amount of about 100 mg, 200 mg, 300 mg, 400 mg, 500 mg, 600 mg, 700 mg, 800 mg, 900 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, 10000 mg, 10500 mg, 11000 mg, 11500 mg, 12000 mg, 12500 mg, 13000 mg, 13500 mg, 14000 mg, 14500 mg, 15000 mg, 16000 mg, 17000 mg, 18000 mg, 19000 mg, 20000 mg, 21000 mg, 22000 mg, 2300 mg, 24000 mg, 25000 mg, 26000 mg, 27000 mg, 28000 mg, 29000 mg, 30000 mg, 31000 mg, 32000 mg, 33000 mg, 34000 mg, 35000 mg, 36000 mg, 37000 mg, 38000 mg, 39000 mg, 40000 mg, 45000 mg, 50000 mg, 55000 mg, 60000 mg, 70000 mg, 80000 mg, 90000 mg, 100000 mg, 110000 mg, 120000 mg, 120000 mg, 130000 mg, 140000 mg, 150000 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, and ±25% for each of the mg values, respectively.

### Embodiment 29

The composition of anyone of embodiments 1-28, wherein the composition comprises curcumin in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, 1000 mg, 1100 mg, 1200 mg, 1200 mg, 1300 mg, 1400 mg, 1500 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, and ±25% for each of the mg values, respectively.

### Embodiment 30

The composition of any one of embodiments 1-29, wherein the composition comprises piperine in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, 1000 mg, 1100 mg, 1200 mg, 1200 mg, 1300 mg, 1400 mg, 1500 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, and ±25% for each of the mg values, respectively.

### Embodiment 31

The composition of anyone of embodiments 1-30, wherein the composition comprises black pepper in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, 1000 mg, 1100 mg, 1200 mg, 1200 mg, 1300 mg, 1400 mg, 1500 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, 24%, and ±25% for each of the mg values, respectively.

### Embodiment 32

The composition of any one of embodiments 1-31, wherein the composition comprises ginger root in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, 1000 mg, 1100 mg, 1200 mg, 1200 mg, 1300 mg, 1400 mg, 1500 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, 24%, and ±25% for each of the mg values, respectively.

### Embodiment 33

The composition of anyone of embodiments 1-32, wherein the composition comprises cardamom in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, 1000 mg, 1100 mg, 1200 mg, 1200 mg, 1300 mg, 1400 mg, 1500 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, 24%, and ±25% for each of the mg values, respectively.

### Embodiment 34

The composition of anyone of embodiments 1-33, wherein the composition comprises cinnamon in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, 1000 mg, 1100 mg, 1200 mg, 1200 mg, 1300 mg, 1400 mg, 1500 mg and being within about at least one of or just one of ±1%, ±2%, ±3%, ±4%, ±5%, ±6%, ±\7%, ±8%, ±9%, ±10%, ±11%, ±12%, ±13%, ±14%, ±15%, ±16%, ±17%, ±18%, ±19%, ±20%, ±21%, ±22%, ±23%, 24%, and ±25% for each of the mg values, respectively.

### Embodiment 35

The composition of anyone of embodiments 1-34, wherein the composition comprises barley malt in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, 1000 mg, 1100 mg, 1200 mg, 1200 mg, 1300 mg, 1400 mg, 1500 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, 24%, and ±25% for each of the mg values, respectively.

### Embodiment 36

The composition of anyone of embodiments 1-35, wherein the composition comprises lecithin in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, 1000 mg, 1100 mg, 1200 mg, 1200 mg, 1300 mg, 1400 mg, 1500 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, 24%, and ±25% for each of the mg values, respectively.

### Embodiment 37

The composition of anyone of embodiments 1-36, wherein the composition comprises cocoa powder in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg-190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, 1000 mg, 1100 mg, 1200 mg, 1200 mg, 1300 mg, 1400 mg, 1500 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, 24%, and ±25% for each of the mg values, respectively.

### Embodiment 38

The composition of any one of embodiments 1-37, wherein the composition comprises chocolate flavoring in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, 1000 mg, 1100 mg, 1200 mg, 1200 mg, 1300 mg, 1400 mg, 1500 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, 24%, and ±25% for each of the mg values, respectively.

### Embodiment 39

The composition of anyone of embodiments 1-38, wherein the composition comprises luohan in an amount of about 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg, 550 mg, 600 mg, 700 mg, 800 mg, 900 mg, 1000 mg, 1100 mg, 1200 mg, 1200 mg, 1300 mg, 1400 mg, 1500 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, 24%, and ±25% for each of the mg values, respectively.

### Embodiment 40

The composition of anyone of embodiments 1-39, wherein the composition comprises a sweetener or a sugar substitute in an amount of about 100 mg, 200 mg, 300 mg, 400 mg, 500 mg, 600 mg, 700 mg, 800 mg, 900 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, 10000 mg, 10500 mg, 11000 mg, 11500 mg, 12000 mg, 12500 mg, 13000 mg, 13500 mg, 14000 mg, 14500 mg, 15000 mg, 16000 mg, 17000 mg, 18000 mg, 19000 mg, 20000 mg, 21000 mg, 22000 mg, 2300 mg, 24000 mg, 25000 mg, 26000 mg, 27000 mg, 28000 mg, 29000 mg, 30000 mg, 31000 mg, 32000 mg, 33000 mg, 34000 mg, 35000 mg, 36000 mg, 37000 mg, 38000 mg, 39000 mg, 40000 mg, 45000 mg, 50000 mg, 55000 mg, 60000 mg, 70000 mg, 80000 mg, 90000 mg, 100000 mg, 110000 mg, 120000 mg, 120000 mg, 130000 mg, 140000 mg, 150000 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, and ±25% for each of the mg values, respectively.

### Embodiment 41

The composition of anyone of embodiments 1-40, wherein the composition comprises mushroom grown in fermented soy, the mushroom provided in an amount of about 100 mg, 200 mg, 300 mg, 400 mg, 500 mg, 600 mg, 700 mg, 800 mg, 900 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, 10000 mg, 10500 mg, 11000 mg, 11500 mg, 12000 mg, 12500 mg, 13000 mg, 13500 mg, 14000 mg, 14500 mg, 15000 mg, 16000 mg, 17000 mg, 18000 mg, 19000 mg, 20000 mg, 21000 mg, 22000 mg, 2300 mg, 24000 mg, 25000 mg, 26000 mg, 27000 mg, 28000 mg, 29000 mg, 30000 mg, 31000 mg, 32000 mg, 33000 mg, 34000 mg, 35000 mg, 36000 mg, 37000 mg, 38000 mg, 39000 mg, 40000 mg, 45000 mg, 50000 mg, 55000 mg, 60000 mg, 70000 mg, 80000 mg, 90000 mg, 100000 mg, 110000 mg, 120000 mg, 120000 mg, 130000 mg, 140000 mg, 150000 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, 24%, and ±25% for each of the mg values, respectively.

### Embodiment 42

The composition of any one of embodiments 1-41, wherein the composition comprises the mushroom species provided in an amount of about 100 mg, 200 mg, 300 mg, 400 mg, 500 mg, 600 mg, 700 mg, 800 mg, 900 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, 10000 mg, 10500 mg, 11000 mg, 11500 mg, 12000 mg, 12500 mg, 13000 mg, 13500 mg, 14000 mg, 14500 mg, 15000 mg, 16000 mg, 17000 mg, 18000 mg, 19000 mg, 20000 mg, 21000 mg, 22000 mg, 2300 mg, 24000 mg, 25000 mg, 26000 mg, 27000 mg, 28000 mg, 29000 mg, 30000 mg, 31000 mg, 32000 mg, 33000 mg, 34000 mg, 35000 mg, 36000 mg, 37000 mg, 38000 mg, 39000 mg, 40000 mg, 45000 mg, 50000 mg, 55000 mg, 60000 mg, 70000 mg, 80000 mg, 90000 mg, 100000 mg, 110000 mg, 120000 mg, 120000 mg, 130000 mg, 140000 mg, 150000 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, 24%, and ±25% for each of the mg values, respectively.

### Embodiment 43

The composition of anyone of embodiments 1-42, wherein the composition comprises protein in an amount of about 100 mg, 200 mg, 300 mg, 400 mg, 500 mg, 600 mg, 700 mg, 800 mg, 900 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, 10000 mg, 10500 mg, 11000 mg, 11500 mg, 12000 mg, 12500 mg, 13000 mg, 13500 mg, 14000 mg, 14500 mg, 15000 mg, 16000 mg, 17000 mg, 18000 mg, 19000 mg, 20000 mg, 21000 mg, 22000 mg, 2300 mg, 24000 mg, 25000 mg, 26000 mg, 27000 mg, 28000 mg, 29000 mg, 30000 mg, 31000 mg, 32000 mg, 33000 mg, 34000 mg, 35000 mg, 36000 mg, 37000 mg, 38000 mg, 39000 mg, 40000 mg, 45000 mg, 50000 mg, 55000 mg, 60000 mg, 70000 mg, 80000 mg, 90000 mg, 100000 mg, 110000 mg, 120000 mg, 120000 mg, 130000 mg, 140000 mg, 150000 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, 24%, and ±25% for each of the mg values, respectively.

### Embodiment 44

The composition of anyone of embodiments 1-43, wherein the composition comprises dietary fiber in an amount of about 100 mg, 200 mg, 300 mg, 400 mg, 500 mg, 600 mg, 700 mg, 800 mg, 900 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, 10000 mg, 10500 mg, 11000 mg, 11500 mg, 12000 mg, 12500 mg, 13000 mg, 13500 mg, 14000 mg, 14500 mg, 15000 mg, 16000 mg, 17000 mg, 18000 mg, 19000 mg, 20000 mg, 21000 mg, 22000 mg, 2300 mg, 24000 mg, 25000 mg, 26000 mg, 27000 mg, 28000 mg, 29000 mg, 30000 mg, 31000 mg, 32000 mg, 33000 mg, 34000 mg, 35000 mg, 36000 mg, 37000 mg, 38000 mg, 39000 mg, 40000 mg, 45000 mg, 50000 mg, 55000 mg, 60000 mg, 70000 mg, 80000 mg, 90000 mg, 100000 mg, 110000 mg, 120000 mg, 120000 mg, 130000 mg, 140000 mg, 150000 mg, 160000 mg, 170000 mg, 180000 mg, 190000 mg, 200000 mg, 210000 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, and ±25% for each of the mg values, respectively.

### Embodiment 45

The composition of anyone of embodiments 1-44, wherein the composition comprises polyunsaturated fat in an amount of about 100 mg, 200 mg, 300 mg, 400 mg, 500 mg, 600 mg, 700 mg, 800 mg, 900 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, 10000 mg, 10500 mg, 11000 mg, 11500 mg, 12000 mg, 12500 mg, 13000 mg, 13500 mg, 14000 mg, 14500 mg, 15000 mg, 16000 mg, 17000 mg, 18000 mg, 19000 mg, 20000 mg, 21000 mg, 22000 mg, 2300 mg, 24000 mg, 25000 mg, 26000 mg, 27000 mg, 28000 mg, 29000 mg, 30000 mg, 31000 mg, 32000 mg, 33000 mg, 34000 mg, 35000 mg, 36000 mg, 37000 mg, 38000 mg, 39000 mg, 40000 mg, 45000 mg, 50000 mg, 55000 mg, 60000 mg, 70000 mg, 80000 mg, 90000 mg, 100000 mg, 110000 mg, 120000 mg, 120000 mg, 130000 mg, 140000 mg, 150000 mg, 160000 mg, 170000 mg, 180000 mg, 190000 mg, 200000 mg, 210000 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, 24%, and ±25% for each of the mg values, respectively.

### Embodiment 46

The composition of anyone of embodiments 1-45, wherein the composition comprises monounsaturated fat in an amount of about 100 mg, 200 mg, 300 mg, 400 mg, 500 mg, 600 mg, 700 mg, 800 mg, 900 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, 10000 mg, 10500 mg, 11000 mg, 11500 mg, 12000 mg, 12500 mg, 13000 mg, 13500 mg, 14000 mg, 14500 mg, 15000 mg, 16000 mg, 17000 mg, 18000 mg, 19000 mg, 20000 mg, 21000 mg, 22000 mg, 2300 mg, 24000 mg, 25000 mg, 26000 mg, 27000 mg, 28000 mg, 29000 mg, 30000 mg, 31000 mg, 32000 mg, 33000 mg, 34000 mg, 35000 mg, 36000 mg, 37000 mg, 38000 mg, 39000 mg, 40000 mg, 45000 mg, 50000 mg, 55000 mg, 60000 mg, 70000 mg, 80000 mg, 90000 mg, 100000 mg, 110000 mg, 120000 mg, 120000 mg, 130000 mg, 140000 mg, 150000 mg, 160000 mg, 170000 mg, 180000 mg, 190000 mg, 200000 mg, 210000 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, 24%, and ±25% for each of the mg values, respectively.

### Embodiment 47

The composition of any one of embodiments 1-46, wherein the composition comprises carbohydrate(s) in an amount of about 100 mg, 200 mg, 300 mg, 400 mg, 500 mg, 600 mg, 700 mg, 800 mg, 900 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, 10000 mg, 10500 mg, 11000 mg, 11500 mg, 12000 mg, 12500 mg, 13000 mg, 13500 mg, 14000 mg, 14500 mg, 15000 mg, 16000 mg, 17000 mg, 18000 mg, 19000 mg, 20000 mg, 21000 mg, 22000 mg, 2300 mg, 24000 mg, 25000 mg, 26000 mg, 27000 mg, 28000 mg, 29000 mg, 30000 mg, 31000 mg, 32000 mg, 33000 mg, 34000 mg, 35000 mg, 36000 mg, 37000 mg, 38000 mg, 39000 mg, 40000 mg, 45000 mg, 50000 mg, 55000 mg, 60000 mg, 70000 mg, 80000 mg, 90000 mg, 100000 mg, 110000 mg, 120000 mg, 120000 mg, 130000 mg, 140000 mg, 150000 mg, 160000 mg, 170000 mg, 180000 mg, 190000 mg, 200000 mg, 210000 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, 24%, and ±25% for each of the mg values, respectively.

### Embodiment 48

The composition of any one of embodiments 1-47, wherein the composition comprises saturated fat in an amount of about 100 mg, 200 mg, 300 mg, 400 mg, 500 mg, 600 mg, 700 mg, 800 mg, 900 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, 10000 mg, 10500 mg, 11000 mg, 11500 mg, 12000 mg, 12500 mg, 13000 mg, 13500 mg, 14000 mg, 14500 mg, 15000 mg, 16000 mg, 17000 mg, 18000 mg, 19000 mg, 20000 mg, 21000 mg, 22000 mg, 2300 mg, 24000 mg, 25000 mg, 26000 mg, 27000 mg, 28000 mg, 29000 mg, 30000 mg, 31000 mg, 32000 mg, 33000 mg, 34000 mg, 35000 mg, 36000 mg, 37000 mg, 38000 mg, 39000 mg, 40000 mg, 45000 mg, 50000 mg, 55000 mg, 60000 mg, 70000 mg, 80000 mg, 90000 mg, 100000 mg, 110000 mg, 120000 mg, 120000 mg, 130000 mg, 140000 mg, 150000 mg, 160000 mg, 170000 mg, 180000 mg, 190000 mg, 200000 mg, 210000 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, and ±25% for each of the mg values, respectively.

### Embodiment 49

The composition of anyone of embodiments 1-44, wherein the composition comprises total fat in an amount of about 100 mg, 200 mg, 300 mg, 400 mg, 500 mg, 600 mg, 700 mg, 800 mg, 900 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, 10000 mg, 10500 mg, 11000 mg, 11500 mg, 12000 mg, 12500 mg, 13000 mg, 13500 mg, 14000 mg, 14500 mg, 15000 mg, 16000 mg, 17000 mg, 18000 mg, 19000 mg, 20000 mg, 21000 mg, 22000 mg, 2300 mg, 24000 mg, 25000 mg, 26000 mg, 27000 mg, 28000 mg, 29000 mg, 30000 mg, 31000 mg, 32000 mg, 33000 mg, 34000 mg, 35000 mg, 36000 mg, 37000 mg, 38000 mg, 39000 mg, 40000 mg, 45000 mg, 50000 mg, 55000 mg, 60000 mg, 70000 mg, 80000 mg, 90000 mg, 100000 mg, 110000 mg, 120000 mg, 120000 mg, 130000 mg, 140000 mg, 150000 mg, 160000 mg, 170000 mg, 180000 mg, 190000 mg, 200000 mg, 210000 mg and being within about at least one of or just one of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±21%, 22%, ±23%, 24%, and ±25% for each of the mg values, respectively.

### Embodiment 50

The composition of anyone of embodiments 1-49, wherein the amounts recited are per serving and each serving is about one scoop of about 20 grams powder if provided in a powdered dosage form.

### Embodiment 51

The compositions of anyone of embodiments 1-50, wherein if the amounts recited exceed the serving size then such amounts are capped at the maximum serving size.

### Embodiment 52

The composition of any one of embodiments 1-51, wherein the mushroom is grown in fermented soy with a bacterial species that produces lactic acid at least during a part of a mushroom growth cycle.

### Embodiment 53

The composition of anyone of embodiments 1-52, wherein the mushroom species is in a dried form, a dried pulverized form, a pulverized form, a dried powder form, a dried and pulverized powder form and a powder form.

### Embodiment 54

The composition of anyone of embodiments 1-53, wherein the composition is provided in the form of a food, a pasta, a ready to use nutritional supplemental optionally containing peanut paste not requiring reconstitution with water.

### Embodiment 55

The composition of anyone of embodiments 1-54, wherein the composition is provided in the form of a food, a pasta, a ready to eat nutritional supplemental optionally containing peanut paste, wherein the ready to eat nutritional supplement does not require reconstitution with water or other liquid and the ready to eat nutritional supplement is provided in a single use disposable or a multiple use reusable container or sealed pouch.

### Embodiment 56

The composition of anyone of embodiments 1-55, wherein the composition optionally further comprises one or more of a vegetable fat, peanuts paste, dry skimmed milk, maltodextrin, lactoserum, sacchorose, a complex of minerals and vitamins, wherein said minerals include one or more of calcium, phosphorus, potassium, magnesium, sodium, iron, zinc, copper, iodine, selenium vitamin A, vitamin B-1, vitamin B-2, vitamin B-6, vitamin B-12, vitamin C, vitamin D, vitamin E, vitamin K, folic acid, calcium-D-pantothenate, biotin, niacin, and combinations or any subcombinations thereof.

### Embodiment 57

The composition of anyone of embodiments 1-56, wherein the composition optionally further comprises one or more of a vegetable fat (about 34-36 gms), peanuts paste (10-15 gms), dry skimmed milk, maltodextrin, lactoserum, saccharose, a complex of minerals and vitamins, wherein said minerals include one or more of calcium (320 mg), phosphorus (390-400 mg), potassium (1000-1100 mg), magnesium (90-100 mg), sodium (180-200 mg), iron (10-20 mg), zinc (10-20 mg), copper (1-2 mg), iodine (100-120 mcg), selenium (1-5 mcg). vitamin A (0.9-1 mg), vitamin B-1 (0.1-0.6 mg), vitamin B-2 (1-2 mg), vitamin B-6 (0.1-1 mg), vitamin B-12 (0.1-1 mcg), vitamin C (10-100 mg), vitamin D (10-20 mcg), vitamin E (10-30 mg), vitamin K (10-2 Smcg), folic acid (200-220 mcg), calcium-D-pantothenate (1-5 mg), biotin (10-70 mcg), niacin (1-10 mg), and combinations or any-sub-combinations thereof, wherein the weight amounts are per sealed satchet (90-100 gm satchet weight) providing a water insoluble paste being ready to eat and essentially devoid of water and resistant to bacgterial contamination even after the sealed satchet is open and wherein each satchet provides approximately 500-550 Kcal per 100 gm satchet.

### Embodiment 58

The composition of anyone of embodiments 1-58, wherein the composition is provided as a therapeutic food in paste form suitable for treating severely malnourished patients, those suffering from endemic gastroenteritis, HIV infection coupled with malnutrition, HIV/AIDS coupled with malnutrition, diarrhea, in a ready to eat form containing essentially no water and suitable for feeding to infants, babies, young children, children, adolescents, teenagers, young adults and the middle aged and elderly persons in need thereof afflicted with or suffering from malnutrition.

### Embodiment 59

A method for treating an individual suffering from one or more diseases, or bacterial infection, viral infection, AIDS, HIV, chronic malnutrition, chronic diarrhea, diarrhea, gastroenteritis, cancer, anorexia, Crohn's disease, ulcerative colitis, type II diabetes, the method comprising the step of: (i) administering a therapeutically effective nutritional amount of a composition of anyone of embodiments 1-58 to an infant (6 months-2 years of age), a baby (2-3 years of age), a young child (3-6 years of age), a child (6-10 years of age), an adolescent (10-13 years of age), a teenager (13-18 years of age), a young adult (18-29 years age), a middle aged adult (30-55 years of age), an elderly person (55-85 years of age), wherein said individual is in need of nutritional supplementation.

### Embodiment 60

The method of embodiment 59, wherein the individual is suffering from severe and chronic malnutrition.

A list of references follows relating to fermented soy.
Klein, A. et al, “Prolonged stabilization of platinum-resistant ovarian cancer in a single patient consuming a fermented soy therapy”, Gynecologic Oncology, vol. 100, pp. 205-9, 2006.
Siddiqui, R. A. et al, “Attenuation of proteolysis and muscle wasting by curcumin c3 complex in MAC16 colon tumour-bearing mice”, British Journal of Nutrition, vol. 102, pp. 967-75, 2009.
Daniells, Stephen, “Spices may boost breast health: Study”, nutraingredients.com, http://www.nutraingredients.com!Research/Spices-may-boost-breast-health-S-tudy, 9 Dec. 2009.
Haelan Research Foundation, “Study Report: Effects of Haelan 951® on Tumor Cell Lines”, www.haelanresearchfoundation.com/Documents/l.pdfuttp://www.haelanresearch-foundation.com/Documents/l.pdf.
Natarajan, K. et al, “Protein Tyrosine Kinase Inhibitors Block Tumor Necrosis Factor-Induced Activation of Nuclear Factor-kB, Degradation of IkBa, Nuclear Translocation of p65, and Subsequent Gene Expression”, Archives of Biochemistry and Biophysics, vol. 352, no. 1, pp. 59-70, 1998.
Xiao Ou Shu et al., “Soy Food Intake and Breast Cancer Survival”, Journal of the American Medical Association, vol. 302, no. 22, pp. 2437-43, 2009.
U.S. Pat. No. 6,756,066 B2.
Holkner, U. et al, “Biotechnological advantages of laboratory-scale solid-state fermentation with fungi”, Appl Microbiol Biotechnol, vol. 64, pp. 175-86, 2004.
Rani, P. et al, “Evaluation of Lignocellulosic Wastes for Production of Edible Mushrooms”, Appl Biochem Biotechnol, vol. 151, pp. 151-9, 2008.
U.S. Pat. No. 6,841,180 B2.
U.S. patent application Ser. No. 11/269,810.
U.S. patent application Ser. No. 11/824,213.
Uhart, Marina et al., “Utilization of new naturally occurring strains and supplementation to improve the biological efficiency of the edible mushroom Agrocybe cylindracea”, J Ind Microbiol Biotechnol, vol. 35, pp. 595-602, 2008.
Kim, S. et al, “Antimutagenic activity of soybeans fermented with basidiomycetes in Ames/Salmonella test”, Biotechnology Letters, vol. 22, pp. 1197-202, 2000.
Kakarala, M. et al, “Targeting breast stem cells with the cancer preventive compounds curcumin and piperine”, Breast Cancer Research and Treatment, 2009.
Nair, Dr. Vijaya & Hernandez, Vic, “Haelan 951: A Novel Fermented Soya Beverage and Safe Nutritive Adjunct in Prostate Cancer Therapies: Is There a Possible Chemopreventive Role?”, Townsend Letter for Doctors and Patients, no. 228, pp. 98-102, 2002.
Nair, Dr. Vijaya, “Soy and Cancer Survivors: Dietary Supplementation with Fermented Soy Nutraceutical, Haelan 951 in Patients Who Survived Terminal Cancers”, Townsend Letter for Doctors and Patients, no. 256, pp. 48-58, 2004.
U.S. Pat. No. 6,346,284 B1.

All patents, all patent applications, all publications, and all references cited herein are incorporated herein by reference in their entirety for all purposes.

The foregoing description, for purpose of explanation, has been described with reference to specific embodiments. However, the illustrative discussions above are not intended to be exhaustive or to limit the presently claimed or described invention(s) to the precise forms disclosed. Many modifications and variations are possible in view of the above teachings. The embodiments were chosen and described in order to best explain the principles of the presently claimed or described invention(s) and its practical applications, to thereby enable others skilled in the art to best utilize the presently claimed or described invention(s) and various embodiments with various modifications as are suited to the particular use(s) contemplated.

Although the present invention describes in detail certain embodiments, it is understood that variation and modifications exist as would be known to those skilled in the art upon reading this application to be within the scope of the invention(s) described or claimed herein. Accordingly, the presently claimed or disclosed invention(s) is/are intended to encompass all such alternatives, modifications and variations that are within their scope and as set forth in one or more embodiments and as set forth in one or more claims.

1. A composition consisting essentially of:
(a) a mushroom selected from the group consisting of Agaricus blazei, Flamulina velutipes, Lentinula edodes, Pleurotus ostreatus, Cordyceps sinensis, Auricularia auricular, Tremella fuciformis, Polyporus umbellatus, Polyporus umbellatus, Coriolus versicolor, Grifola frondosa, Ganoderma lucidum, lnonotus obliquus, Schizophylum commune, Hericium erinaceus, Poria cocos, Phellinus igniarius, Agrocybe cylindracea, A. bisporus, Volvariella volvacea, Agrocybe aegerita, Hypsizygus marmoreus, Lepista nuda, Coprinus comatus, Pholiota nameko, and Elfvingia applanata,
b) fermented soy; and
(c) a curcuminoid selected from the group consisting of curcumin, demethoxycurcumin, or bis-demethoxycurcumin.

2. The composition of claim 1, wherein the soy is fermented with one or more of:
(c) Lactobacillus bulgaricus,
(d) Lactobacillus casei,
(e) Lactobacillus plantarum,
(f) Streptococcus thermophilus,
(g) Bifidobacterium bifidum,
(h) Bifidobacterium breve,
(i) Bifidobacterium infantis,
(j) Bifidobacterium longum,
(k) Bifidobacterium lactis,
(l) Lactobacillus acidophilus,
(m) Lactobacillus helveticus,
(n) Lactobacillus rhamnosus,
(o) Lactobacillus salviarious,
(p) Lactobacillus johnsonii,
(q) Lactobacillus jermentum,
(r) Lactobacillus lactis,
(s) Lactobacillus sanfranciscensis,
(t) Pediococcus acidiiactici,
(u) Streptococcus faccium,
(v) B. Streptococcus thermophilus,
(w) a probiotic bacteria, or
(x) a prebiotic bacteria.

3. The composition of claim 1, wherein the mushroom is a dried mushroom.

4. The composition of claim 1, wherein the mushroom is a powdered mushroom.

5. The composition of claim 1 further consisting essentially of piperine from Piper nigrum, Piper longum, or a combination thereof.

6. The composition of claim 5, wherein the piperine has a purity of at least 95%.

7. The composition of claim 1, wherein the mushroom species is one or more of: Ganoderma spp., Stropharia spp., or Cordyceps spp.

8. The composition of claim 1 further consisting essentially of a member selected from the group consisting of: dietary fiber, protein, fruit(s), vegetable(s), spice(s), botanical plant part(s), botanical plant extracts, tuberous material, vitamin(s), mineral(s), anti-oxidants(s), ocean herb(s), flavoring agent(s), sweetener(s), anthocyanin(s), carbohydrate(s), glucosinolate(s), isoflavone(s), oligomeric proanthocyanidin(s), polyphenol(s), punicalagin(s), essential monosaccharides(s), and combinations thereof.

9. The composition of claim 1 further consisting essentially of a member selected from the group consisting of: daidzein, genistein, daidzin, genistin, miso, natto, ginger, ginger root, tea, green tea, coffee, cultured soy, lactic acid, turmeric, cardamom, black pepper, water, sparkling ginger, vanilla flavor, coffee flavor, anti-emetic, coke syrup, soybean, black soybean, corn, wild blueberry, acai, blackcurrant, sweet cherry, raspberry, elderberry, blackberry, aronia, wild bilberry, red wine powder, red wine extract, pomegranate, blackcurrants, cranberries, cherry powder, cherry extract, wild blueberry extract, wild blueberries, grapes, grape seed, raspberry seed, cranberries, prunes, cherries, strawberries, cranberry, grape seed extract, citric acid, mangosteen, acerola, camu camu, whole coffee fruit, coffee extracts and powders, mango, pineapple, noni powder, grapefruit, lemon, tomato, spinach, aloe vera, peas, alfalfa, garlic, nettles, grain-based fibers, grains, adzuki, barley bran, oats, 55% oat-soluble freeze-dried fiber, beta-glucans, powdered sprout concentrate, blends of broccoli, water cress, daikon radish, alfalfa, amaranth, cabbage, kale, mustard, onions, flax seed, red clover, cauliflower sprouts, winter cherry, licorice, giant potato, white musale, indian gooseberry, aloeweed, elephant creeper, black musale, caper plant, indian gum arabic tree, amla, pedalium, indian timospora, cinnamon, nutmeg, cloves, aniseed, corn, beet, banana, aronia, apple, orange, acerola, peanut, rice, carrot, bean sprouts, allicin, allin, beta carotene, boron, chromium, ellagic acid, ellagitannin, lutein, lycopene, quercitin, quinic acid, reservatol, water soluble fiber, water soluble beta glucans, sulforaphane, vitamin A, vitamin C, vitamin D, vitamin E, vitamin K, vitamin B-1, vitamin B-2, vitamin B-3, vitamin B-6, vitamin B-12, folate, folic acid, calcium, phosphorus, potassium, magnesium, sodium, iron, zinc, copper, iodine, selenium, lecithin, soy lecithin, cocoa powder, chocolate, chocolate flavoring, luohan, ascorbic acid, mannose, fucose, xylose, galactose, glucose, N-acetylneuraminic acid, N-acetylgalactosamine, N-acetylglucosamine, vegetable fat, peanut paste, dry skimmed milk, maltodextrin, calcium-D-pantothenate, biotin, niacin, lactoserum, saccharose, and combinations thereof.

10. The composition of claim 1 provided in one or more forms selected from the group consisting of: beverage, capsule, tablet, caplet, emulsion, suspension, syrup, solid dosage form, liquid dosage form, food, cosmetic, and combinations thereof.

11. The composition of claim 1 further consisting essentially of one or more fruit powders.

12. The composition of claim 1 further consisting essentially of one or more ingredients selected from the group consisting of: ascorbic acid, ashwagandha, barley, barley malt, carotene, beta-carotene, bilberry, piperine, and combinations thereof.

13. The composition of claim 1, wherein the composition further consists essentially of one or more of: a vegetable fat in an amount from about 34 g to about 36 g; peanut paste in an amount from about 10 g to about 15 g; dry skimmed milk; maltodextrin; lactoserum; sacchorose; a complex of minerals and vitamins, wherein said minerals include one or more of calcium (320 mg), phosphorus (390-400 mg), potassium (1000-1100 mg), magnesium (90-100 mg), sodium (180-200 mg), iron (10-20 mg), zinc (10-20 mg), copper (1-2 mg), iodine (100-120 mcg), selenium (1-5 mcg). vitamin A (0.9-1 mg), vitamin B-1 (0.1-0.6 mg), vitamin B-2 (1-2 mg), vitamin B-6 (0.1-1 mg), vitamin B-12 (0.1-1 mcg), vitamin C (10-100 mg), vitamin D (10-20 mcg), vitamin E (10-30 mg), vitamin K (10-25 mcg), folic acid (200-220 mcg), calcium-D-pantothenate (1-5 mg), biotin (10-70 mcg), niacin (1-10 mg); and combinations thereof, wherein the weight amounts are per sealed satchet (90-100 g satchet weight) provide a water insoluble paste being ready to eat and essentially devoid of water and resistant to bacterial contamination even after the sealed satchet is open, and wherein each satchet provides approximately 500-550 Kcal per 100 g satchet.

14. A method for treating a disease condition or a disease symptom in an individual suffering from the disease, comprising administering to the individual in need of such treatment a therapeutically effective amount of a composition according to claim 1, wherein the disease is selected from the group consisting of breast cancer, lung cancer, prostate cancer, leukemia, cancer, recurrence of cancer, cardiovascular disease, adult onset diabetes, Crohn's disease, irritable bowel syndrome, ulcerative colitis, skin rash, psoriasis, atopic dermatitis, arthritis, tumor metastasis, tumor growth, infection, rheumatism, diabetic wound, runny nose, cough, liver disorder, cirrhosis, precancerous cancer progression, jaundice, Alzheimer's disease, menopause, symptoms of menopause, inflammation, severe malnutrition, malnutrition with HIV infection, HIV condition, HIV/AIDS with malnutrition, endemic gastroenteritis, diarrhea, an underweight condition, and an immune compromised condition.

15. The method of claim 14, wherein the composition is provided as a therapeutic food in paste form that is ready to eat and containing essentially no water.

16. A method of treating an individual suffering from one or more diseases, selected from bacterial infection, viral infection, AIDS, HIV, chronic malnutrition, chronic diarrhea, diarrhea, gastroenteritis, cancer, anorexia, Crohn's disease, ulcerative colitis, or type II diabetes, the method comprising the step of: administering a therapeutically effective nutritional amount of a composition of claim 1 to an individual in need thereof.

17. The method of claim 16, wherein the individual is suffering from severe and chronic malnutrition.

18. The composition of claim 9, wherein the beverage is selected from the group consisting of smoothies, specialty beverages, carbonated beverages, energy beverages, functional beverages, diet beverages, shakes, green drinks, antioxidant beverages, weight management beverages, meal replacement beverages, lemonade, tropical beverages, electrolytic beverages, herbal tea beverages, teas, and coffees.

19. The composition of claim 10, wherein the food is selected from the group consisting of soup, nutritional bar, snack, cereal, breads, cake, dessert, spread, salad dressing, pet food, cooked food, ice cream, yogurt, meal replacement paste, combination with protein from peanuts, combination with peanut oil, combination with canola oil, combination with olive oil, and combinations thereof.

20. The composition of claim 11, wherein the fruit powder is selected from the group consisting of: acai powder, apple powder, apricot powder, chokeberry powder, aronia powder, banana powder, bilberry powder, blackberry powder, blackcurrant powder, wild blueberry powder, blueberry powder, tart cherry powder, sweet cherry powder, cranberry powder, white cranberry powder, elderberry powder, wolfberry powder, grape powder, grapefruit powder, kiwi powder, lemon powder, lychee powder, mango powder, mangosteen powder, maqui powder, noni powder, orange powder, peach powder, pear powder, persimmon powder, pineapple powder, plum powder, pomegranate powder, prune powder, raspberry powder, black raspberry powder, raspberry seed powder, strawberry powder, strawberry seed powder, watermelon powder, goji powder, yum berry powder, and combinations thereof.
